(19) United States
(12) Patent Application Publication
(10) Pub. No.: US 2025/0277008 A1
(43) Pub. Date: Sep. 4, 2025

(54) Novel Anti-Bacterial Peptides Derived From Differentiating Stem Cells

(71) Applicant: AGENCY FOR SCIENCE, TECHNOLOGY AND RESEARCH, Singapore (SG)

(72) Inventors: Smarajit Chakraborty, Singapore (SG); Vikashini Ravikumar, Singapore (SG); Shigeki Sugii, Singapore (SG)

(21) Appl. No.: 18/863,724

(22) PCT Filed: May 8, 2023
(86) PCT No.: PCT/SG2023/050319

§ 371 Date: Nov. 7, 2024

(30) Foreign Application Priority Data
May 9, 2022 (SG) 10202204848T

(51) Int. Cl. C07K 14/47; A61K 38/00

(52) U.S. Cl. CPC C07K 14/47; A61K 38/00

(57) ABSTRACT

This technology relates to anti-bacterial peptides and method of treating infections using the peptides.

### FIELD OF THE INVENTION

The present invention relates generally to the field of cell biology. In particular, the present invention relates to anti-bacterial peptides.

### BACKGROUND

Chronic wound management is a large and growing problem due to the aging population, and the sharp rise in the incidence of diabetes and obesity worldwide. It is estimated that 1-2% of the global population experience chronic wound during their lifetime. In the United States, chronic wounds affect around 6.5 million patients with a reported annual spending of US$25 billion. Chronic wounds are primarily due to the occurrence of nosocomial infection that complicates skin lesions. According to National Institute for Health and Clinical Excellence (NICE) (2008), an estimate of 5% of all surgical procedures in the UK result in nosocomial infection, accounting for one in every seven cases. The occurrence of chronic wound infection is greater in diseases such as diabetes mellitus that are predisposed to bacterial infections. An exemplary implication of chronic wound infection is the diabetic foot ulcer (DFU) and diabetic foot infections (DFI) which affects approximately 15% of all patients and are the most common risk factor for lower extremity amputation. In some cases, pathogens, necrotic tissue, and the extracellular matrix (ECM) secreted by the pathogens form into a membranous layer termed biofilm, which are in particular difficult to manage due to the presence of the protective matrix barrier.

As of today, antibiotics stand as the first line of treatment against wound infections, for example, in the form of wound dressings to facilitate tissue regeneration. However, none of them actively manage chronic infections beyond the use of limited choices of existing antibiotics. Chronic wound causing pathogens are predominantly multidrug resistant (MDR), which can easily survive standard antibiotics treatments by gaining anti-microbial resistance (AMR). World health organization (WHO) has declared anti-microbial resistance (AMR) as one of the top 10 global public health threats, which is predicted to kill 10 million people each year by 2050, surpassing cancer with an estimated to cost to the global economy 100 trillion USD. The emergence of antibiotic resistant infections accompanied with the dearth of new antibiotics possess the biggest challenge.

Attempts to resolve this problem such as combination therapies of either anti-microbial peptides (AMPs) or polymers with antibiotics have garnered much attention to treat infections caused by multidrug resistant (MDR) bacterial strains. Although combination therapies have shown favorable effects in in vitro and animal studies, clinical data have been conflicting. In addition, adverse effects have been reported such as nephrotoxicity and ototoxicity, potentially leading to drug interactions and further toxicities in patients receiving multiple medications. Besides, use of combinatorial anti-microbial therapies to treat multidrug resistant (MDR) strains leads to increased anti-microbial resistance (AMR) in pathogens and additional cost burden to the patients. Alternative methods such as skin grafting are considered as gold standards in chronic wound management. However, skin grafting is limited by the quality and quantity of donor skin, which does not solve the problem of bacterial clearance either. Similarly, other new procedures such as delivery of angiogenesis-inducing growth factors and cytokines and hyperbaric oxygen therapy do not tackle with the fundamental issue of infections.

Therefore, what is needed is novel anti-microbial agent effective in treating chronic infections, and method of treating chronic infections using the anti-microbial agent. Furthermore, other desirable features and characteristics will become apparent from the subsequent detailed description and the appended claims, taken in conjunction with the accompanying drawings and this background of the disclosure.

### SUMMARY OF INVENTION

In one aspect, the present disclosure provides an anti-bacterial peptide consisting of the sequence of KVNQIGSVTESIQACKLAQSN (SEQ ID NO: 3).

In one aspect, the present disclosure provides an anti-bacterial peptide consisting of the sequence of HHKAEKSSVLKSKEESH (SEQ ID NO: 4).

In one aspect, the present disclosure provides an anti-bacterial peptide consisting of the sequence of KQRNKVKKIYLDEKR (SEQ ID NO: 5).

In one aspect, the present disclosure provides an anti-bacterial peptide comprising a partial sequence of ENO3, wherein the peptide comprises at least the sequence of KVNQIGSVTESIQACKLAQSN (SEQ ID NO: 3).

In one aspect, the present disclosure provides an anti-bacterial peptide comprising a partial sequence of SPARCL1, wherein the peptide comprises at least a sequence of HHKAEKSSVLKSKEESH (SEQ ID NO: 4).

In one aspect, the present disclosure provides an anti-bacterial peptide comprising a partial sequence of SPARCL1, wherein the peptide comprises at least a sequence of KQRNKVKKIYLDEKR (SEQ ID NO: 5).

In one aspect, the present disclosure provides a conditioned cell culture media (CM) comprising anti-bacterial peptides of claims 1-8, a full length ENO3 peptide, or a full length SPARCL1 peptide.

In one aspect, the present disclosure provides a composition comprising one or more of the anti-bacterial peptides as disclosed herein, a full length ENO3 peptide, or a full length SPARCL1 peptide.

In one aspect, the present disclosure provides a pharmaceutical composition comprising the peptide as disclosed herein, the conditioned cell culture media as disclosed herein, or the composition as disclosed herein.

In one aspect, the present disclosure provides a wound dressing comprising the peptide as disclosed herein, the conditioned cell culture media (CM) as disclosed herein, the composition as disclosed herein, or the pharmaceutical composition as disclosed herein.

In one aspect, the present disclosure provides an in vitro method of inducing anti-bacterial responses in tissue-derived stem cells, wherein the method comprises: (a) obtaining a tissue sample from a subject; (b) isolating tissue-derived stem cells from the tissue sample of (a) in a cell culture; (c) culturing the tissue-derived stem cells in differentiation media to obtain intermediately differentiating cells; and (d) incubating the intermediately differentiating cells with a bacteria culture or a bacterial surface antigen to induce an anti-bacterial response of the intermediately differentiating cells.

In one aspect, the present disclosure provides an in vitro method of inducing anti-bacterial responses in adipose-derived mesenchymal stem cells (ASCs), comprising: (a) obtaining an adipose tissue sample from a subject; (b) isolating adipose-derived mesenchymal stem cells (ASCs) from the adipose tissue sample of (a) in a cell culture; (c) culturing the adipose-derived mesenchymal stem cells (ASCs) in adipogenic differentiation media to obtain intermediately differentiating ASCs; and (d) incubating the intermediately differentiating ASCs with a bacteria culture or a bacterial surface antigen to induce an anti-bacterial response by the intermediately differentiating ASCs.

In one aspect, the present disclosure provides an in vitro method of producing the conditioned cell culture media (CM) as disclosed herein, comprising: (a) obtaining a tissue sample from a subject; (b) isolating tissue-derived stem cells from the tissue sample of (a); (c) culturing the tissue-derived stem cells in a differentiation media to obtain intermediately differentiating stem cells; (d) incubating the intermediately differentiating cells in a cell culture media with a bacteria culture or a bacterial surface antigen; and (e) collecting the conditioned cell culture media (CM) by removing the bacteria and the intermediately differentiating cells.

In one aspect, the present disclosure provides a method of making the conditioned cell culture media (CM) as disclosed herein, comprising: (a) obtaining an adipose tissue sample from a subject; (b) isolating adipose-derived mesenchymal stem cells (ASCs) from the adipose tissue of (a) in a cell culture; (c) culturing the adipose-derived mesenchymal stem cells (ASCs) in adipogenic differentiation media to obtain intermediately differentiating ASCs; (d) incubating the intermediately differentiating ASCs in a cell culture media with a bacteria culture or a bacterial surface antigen; and (e) collecting the conditioned cell culture media (CM) by removing the bacteria and the intermediately differentiating ASCs.

In one aspect, the present disclosure provides a method of treating an infection in a subject, or a method of promoting wound healing and tissue regeneration in a subject, or a method of reducing/preventing formation of biofilm in a subject, comprises administering to the subject a pharmaceutically effective amount of the peptide as disclosed herein, the conditioned cell culture media (CM) as disclosed herein, the composition as disclosed herein, or the pharmaceutical composition as disclosed herein.

In one aspect, the present disclosure provides a nucleic acid composition encoding the peptide as disclosed herein.

In one aspect, the present disclosure provides an expression vector comprising the nucleic acid composition as disclosed herein.

In one aspect, the present disclosure provides a host cell comprising one or more of the nucleic acid compositions as disclosed herein, or one or more of the expression vectors as disclosed herein.

### BRIEF DESCRIPTION OF THE DRAWINGS

FIG. 1A provides a summary of the optimization of the conditioned media culturing conditions for triggering anti-bacterial response, in particular antibacterial response in adipose-derived mesenchymal stem cells (ASCs) based on the parameters of: (i) priming of adipose-derived mesenchymal stem cells (ASCs) with either heat-inactivated bacteria or live bacteria at the multiplicity of infection (MOI) ratios of 1:2, 1:20, 1:200, or 1:2000 (Bacteria:ASC) as indicated in FIG. 1A; (ii) priming period of 3, 6, 12 or 24 hours; (iii) stages of adipose-derived mesenchymal stem cells (ASCs) undergoing adipogenic differentiation as indicated based on the number of dates after differentiation (0 day, 6 days, or 12 days); (iv) culturing conditions in presence of either high or low glucose and in the presence or absence of 10% FBS. Conditions showing >50% bacterial killing compared to media control are circled. As shown in the summary, an exemplary condition for inducing anti-bacterial response in mesenchymal stem cells comprises using live bacterial of a multiplicity of infection (MOI) ratio of 1:200 (bacteria:ASC, for example), under either high or low glucose levels, or in the presence or absence of FBS. The condition further comprises co-culturing the bacterial and the mesenchymal stem cells are co-cultured for about 24 hours, wherein the mesenchymal stem cells are intermediately differentiating stem cells.

FIG. 1B summaries the various combinations of the conditions tested in FIG. 1A.

FIG. 1C shows the viability of exemplary bacterium Salmonella typhimurium (ST) treated with the conditioned media obtained from culturing adipose derived mesenchymal stem cell line from healthy donors (ATCC SCRC 4000) under the conditions summarized in FIG. 1A. The percentage of Colony Forming Units (CFU) counts of the treated bacteria are shown for each conditions stratified based on the stem cell differentiation stages, and the strain of priming bacteria used. As demonstrated in FIG. 1C, intermediately differentiating stem cells (D6: 6 days) primed with S. typhimurium (ST) or Pseudomonas aeruginosa (PA) show lower than 50% S. typhimurium (ST) Colony Forming Units (CFU) counts, indicating stronger anti-bacterial activity compared to other conditions against exemplary bacterium S. typhimurium (ST).

FIG. 1D shows the viability of exemplary bacterium P. aeruginosa (PA) treated with the conditioned media obtained from culturing adipose derived mesenchymal stem cell line from healthy donors (ATCC SCRC 4000) under the conditions summarized in FIG. 1A. The percentage of Colony Forming Units (CFU) counts of the treated bacteria are shown for each conditions stratified based on the stem cell differentiation stages, and the strain of priming bacteria used. As demonstrated in FIG. 1D, intermediately differentiating stem cells (D6: 6 days) primed with S. typhimurium (ST) or P. aeruginosa (PA) show lower than 50% P. aeruginosa (PA) Colony Forming Units (CFU) counts, indicating stronger anti-bacterial activity compared to other conditions against exemplary bacterium P. aeruginosa (PA).

FIG. 1E shows the viability of exemplary bacterium S. aureus (SA) treated with the conditioned media obtained from culturing adipose derived mesenchymal stem cell line from healthy donors (ATCC SCRC 4000) under the conditions summarized in FIG. 1A. The percentage of Colony Forming Units (CFU) counts of the treated bacteria are shown for each conditions stratified based on the stem cell differentiation stages, and the strain of priming bacteria used. As demonstrated in FIG. 1E, intermediately differentiating stem cells (D6: 6 days) primed with S. typhimurium (ST) or P. aeruginosa (PA) show lower than 50% S. aureus (SA) Colony Forming Units (CFU) counts, indicating stronger anti-bacterial activity compared to other conditions against exemplary bacterium S. aureus (SA).

FIG. 1F shows the viability of exemplary bacterium S. typhimurium (ST) treated with the conditioned media obtained from culturing adipose derived mesenchymal stem cell line from obese patients (S29) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1F, intermediately differentiating stem cells (D6: 6 days) primed with S. typhimurium (ST) or P. aeruginosa (PA) show lower than 50% S. typhimurium (ST) Colony Forming Units (CFU) counts, indicating stronger anti-bacterial activity compared to other conditions against exemplary bacterium S. typhimurium (ST).

FIG. 1G shows the viability of exemplary bacterium P. aeruginosa (PA) treated with the conditioned media obtained from culturing adipose derived mesenchymal stem cell line from obese patients (S29) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1G, intermediately differentiating stem cells (D6: 6 days) primed with S. typhimurium (ST) or P. aeruginosa (PA) show lower than 50% P. aeruginosa (PA) Colony Forming Units (CFU) counts, indicating stronger anti-bacterial activity compared to other conditions against exemplary bacterium P. aeruginosa (PA).

FIG. 1H shows the viability of exemplary bacterium S. aureus (SA) treated with the conditioned media obtained from culturing adipose derived mesenchymal stem cell line from obese patients (S29) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1H, intermediately differentiating stem cells (D6: 6 days) primed with S. typhimurium (ST) or P. aeruginosa (PA) show lower than 50% S. aureus (SA) Colony Forming Units (CFU) counts, indicating stronger anti-bacterial activity compared to other conditions against exemplary bacterium S. aureus (SA).

FIG. 1I shows the viability of exemplary bacterium S. typhimurium (ST) treated with the conditioned media obtained from culturing adipose derived mesenchymal stem cell line from obese patients (S23) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1I, intermediately differentiating stem cells (D6: 6 days) primed with S. typhimurium (ST) or P. aeruginosa (PA) show lower than 50% S. typhimurium (ST) Colony Forming Units (CFU) counts, indicating stronger anti-bacterial activity compared to other conditions against exemplary bacterium S. typhimurium (ST).

FIG. 1J shows the viability of exemplary bacterium P. aeruginosa (PA) treated with the conditioned media obtained from culturing adipose derived mesenchymal stem cell line from obese patients (S23) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1J, intermediately differentiating stem cells (D6: 6 days) primed with S. typhimurium (ST) or P. aeruginosa (PA) show lower than 50% P. aeruginosa (PA) Colony Forming Units (CFU) counts, indicating stronger anti-bacterial activity compared to other conditions against exemplary bacterium P. aeruginosa (PA).

FIG. 1K shows the viability of exemplary bacterium S. aureus (SA) treated with the conditioned media obtained from culturing adipose derived mesenchymal stem cell line from obese patients (S23) under the conditions summarized in FIG. 1A. The percentage of Colony Forming Units (CFU) counts of the treated microbial are shown for each conditions stratified based on the stem cell differentiation stages, and the strain of priming bacteria used. As demonstrated in FIG. 1K, intermediately differentiating stem cells (D6: 6 days) primed with S. typhimurium (ST) or P. aeruginosa (PA) show lower than 50% S. aureus (SA) Colony Forming Units (CFU) counts, indicating stronger anti-bacterial activity compared to other conditions against exemplary bacterium S. aureus (SA). All of the bacteria strains of FIG. 1C-FIG. 1K are grown in accordance with the Clinical and Laboratory Standards Institute (CLSI) protocol in the presence of bacteria-primed or unprimed conditioned media from adipose derived Mesenchymal stem cell lines from healthy or obese patients. Each value is the mean±SD (n=3). All statistical significance was assessed by using Student's paired t-test, compared to the media control normalized to 100%, *p<0.05.

FIG. 2A provides crystal violet (CV) staining of 3-day old P. aeruginosa (PA) to quantify the presence of biofilms in response to the treatment of different adipose derived mesenchymal stem cell line conditioned media (ASC-CM) as indicated. As shown in FIG. 2A, conditioned media from intermediate adipogenic differentiation stage primed with S. typhimurium (ST) or P. aeruginosa (PA) demonstrate significantly disruption to the biofilm forming abilities of P. aeruginosa (PA) strain.

FIG. 2B provides crystal violet (CV) staining of 3-day old S. typhimurium (ST) to quantify the presence of biofilms in response to the treatment of different adipose derived mesenchymal stem cell line conditioned media (ASC-CM) as indicated. Consistent with the conclusion from FIG. 2A, in FIG. 2B, conditioned media from intermediate adipogenic differentiation stage primed with S. typhimurium (ST) or P. aeruginosa (PA) demonstrate significant disruption to the biofilm forming abilities of S. typhimurium (ST) strain. Each value is the mean±SEM (n=3, biological replicates) for both FIG. 2A and FIG. 2B. All statistical significance was assessed by using Student's paired t-test, *p<0.05.

FIG. 3A shows the generation of an exemplary drug resistant microbial strain by serial passaging of S. typhimurium (ST) with Gentamicin. Upon 16 days of Gentamycin treatment, the Minimum Inhibitory Concentration (MIC) of Gentamycin reached as high as 1024 μg/ml. The drug resistant S. typhimurium (ST) bacterial strain is later used for testing of the conditioned media (CM) obtained from adipose derived mesenchymal stem cell line (ASC).

FIG. 3B provides the viability of the high Minimum Inhibitory Concentration (MIC) (1024 μg/ml) S. typhimurium (ST) bacterial strain (percentage of Colony Forming Units (CFU)) after treatment with adipose derived mesenchymal stem cell line conditioned media (ASC-CM). Conditioned media obtained from intermediately differentiating mesenchymal stem cell culture primed with either S. typhimurium (ST) or P. aeruginosa (PA) effectively killed drug resistant S. typhimurium (ST) strain. Each value is the mean±SD (n=3). All statistical significance was assessed by using Student's paired t-test, *p<0.05.

FIG. 3C further demonstrates the long-term anti-bacterial effect against drug resistant S. typhimurium (ST) bacterial strain of P. aeruginosa (PA) primed ATCC SCRC 4000-conditioned medium. The conditioned medium exhibits prolonged inhibition for at least 28 days without occurrence of resistance in serially passaged drug resistant S. typhimurium (ST) strain based on intracellular ATP levels, which quantifies the viability of the drug resistant S. typhimurium (ST) bacterial strains.

FIG. 4A shows the viability test results of S. typhimurium (ST) in response to HPLC purified vials obtained from the intermediately differentiating adipose derived mesenchymal stem cell line conditioned media (ASC-CM) from obese patient sample S29 to identify the fractions containing the active anti-bacterial agent. Sterile HPLC fractions of the conditioned media are compared against media fractions. In all the 3 conditioned media fractions, vial No. 3 (V3) fraction of either S. typhimurium (ST) or P. aeruginosa (PA) primed conditioned media shows significant anti-bacterial effects based on colony counts against S. typhimurium (ST) compared to other vials. Therefore, the active anti-bacterial agent is likely present in V3.

FIG. 4B shows the viability test results of S. typhimurium (ST) in response to HPLC purified vials obtained from the intermediately differentiating adipose derived mesenchymal stem cell line conditioned media (ASC-CM) from healthy subject sample ATCC SCRC 4000 to identify the fractions containing the active anti-bacterial agent. In agreement with the findings from FIG. 4A, vial No. 3 (V3) fraction of either S. typhimurium (ST) or P. aeruginosa (PA) primed conditioned media shows significant anti-bacterial effects based on colony counts against S. typhimurium (ST) compared to other vials. The active anti-bacterial agent is likely to present in V3.

FIG. 4C shows the viability test results of S. typhimurium (ST) in response to HPLC purified vials obtained from the intermediately differentiating adipose derived mesenchymal stem cell line conditioned media (ASC-CM) from obese patient sample S23 to identify the fractions containing the active anti-bacterial agent compared to media. In line with the results from FIG. 4A and FIG. 4B, the same via No. 3 demonstrates anti-bacterial activities based on the percentage of Colony Forming Units (CFU %), which is not present in other vials. Each value is the mean±SD (n=3). All statistical significance was assessed by using Student's paired t-test, *p<0.05. Based on the consistent results from FIG. 4A, FIG. 4B, and FIG. 4C, active anti-bacterial agents are likely present in the fraction of vial No. 3, which may be further isolated and identified using mass spectrometry. Indeed, from the mass spectrometry analysis, novel peptides are identified.

FIG. 5A provides a verification of the anti-bacterial peptides ENO3 and SPARCL1 identified from the mass spectrometry analysis. FIG. 5A shows the percentage of Colony Forming Units (CFU %) of S. typhimurium (ST) in response to ATCC SCRC 4000 conditioned media pre-treated with antibodies against ENO3 and SPARCL1, and a control peptide LL-37 which is known to have anti-bacterial effects. Conditioned media treated with anti-ENO3 or anti-SPARCL1 fail to inhibit the S. typhimurium (ST) bacterial growth compared to untreated media. Anti-LL-37 treated conditioned media continues to inhibit the growth of S. typhimurium (ST) bacteria culture. Therefore, ENO3 and SPARCL1 are shown to be responsible for the anti-bacterial activity in the adipose derived mesenchymal stem cell line conditioned media (ASC-CM).

FIG. 5B provides the verification of the anti-bacterial peptides ENO3 and SPARCL1 identified from conditioned media obtained from stem cells derived from obese patient S29.

FIG. 5C provides the verification of the anti-bacterial peptides ENO3 and SPARCL1 identified from conditioned media obtained from stem cells derived from obese patient S23. Isotype antibodies are used as negative control. FIG. 5B and FIG. 5C demonstrated consistent outcome as FIG. 5A, confirming the anti-bacterial activity of ENO3 and SPARCL1 identified from the conditioned media.

FIG. 6A tests the anti-bacterial activity of ENO3 at various concentrations against exemplary bacteria strain S. typhimurium (ST). The percentage of Colony Forming Units (CFU %) of S. typhimurium (ST) treated with ENO3 of concentrations ranging from 0 (media) to 2.04 nM are shown. It can be seen that ENO3 shows more than 50% bacterial reduction at the concentration of 2.04 nM.

FIG. 6B tests the anti-bacterial activity of ENO3 at various concentrations against exemplary bacteria strain P. aeruginosa (PA). ENO3 demonstrates more than 50% bacterial reduction of P. aeruginosa (PA) at a low concentration of 0.204 nM.

FIG. 6C tests the anti-bacterial activity of ENO3 at various concentrations against exemplary bacteria strain S. aureus (SA). ENO3 demonstrates more than 50% bacterial reduction of P. aeruginosa (PA) at even lower concentration of 0.102 nM. All of the bacterial strains used in FIG. 6A-FIG. 6C are grown as per CLSI protocol. Based on FIGS. 6A-6C, ENO3 shows anti-bacterial activity against multiple microbial strains at concentrations as low as 0.102 nM.

FIG. 6D tests the anti-bacterial activity of SPARCL1 at various concentrations against exemplary bacteria strain S. typhimurium (ST). SPARCL1 demonstrates more than 50% bacterial reduction of S. typhimurium (ST) at a concentration of 0.0067 nM.

FIG. 6E provides the anti-bacterial activity of SPARCL1 at various concentrations against exemplary bacteria strain P. aeruginosa (PA). SPARCL1 demonstrates more than 50% bacterial reduction of P. aeruginosa (PA) at a concentration of 268 nM.

FIG. 6F provides the anti-bacterial activity of SPARCL1 at various concentrations against exemplary bacteria strain S. aureus (SA). SPARCL1 demonstrates more than 50% bacterial reduction of S. typhimurium (ST) at a concentration of 1000 nM. All tested bacterial strains are grown as per CLSI protocol. Each value is the mean±SD (n=3). All statistical significance was assessed by using Student's paired t-test, *p<0.05. Based on FIGS. 6A-6C, SPARCL1 shows anti-bacterial activity against multiple microbial strains at concentrations as low as 0.0067 nM.

FIG. 6G shows the viable colonies of exemplary bacteria strain S. aureus (SA) after treatment with ENO3. The MIC and MBC values for ENO3 against S. aureus (SA) are both 2 μM.

FIG. 6H shows the viable colonies of exemplary bacteria strain P. aeruginosa (PA) after treatment with ENO3. The MIC value for ENO3 against P. aeruginosa (PA) is 4 μM and the MBC value for ENO3 against P. aeruginosa (PA) is 6 μM.

FIG. 7A shows the anti-biofilm effects of ENO3 at various concentrations. Crystal violet (CV) staining of 3-day old P. aeruginosa (PA) biofilm is shown in response to nM ranges of ENO3 treatment. The P. aeruginosa (PA) biofilm shows disruption of nearly 50% upon treatment with ENO3 at about 0.0102 nM concentration.

FIG. 7B shows the anti-biofilm effects of ENO3 at various concentrations. Crystal violet (CV) staining of 3-day old S. typhimurium (ST) biofilm in response to nM ranges of ENO3 treatment. The S. typhimurium (ST) biofilm shows more than 50% disruption upon treatment with ENO3 at about 0.0102 nM concentration.

FIG. 7C shows the anti-biofilm effects of SPARCL1 at various concentrations. Crystal violet (CV) staining of 3-day old P. aeruginosa (PA) biofilm is shown in FIG. 7C in response to nM ranges of SPARCL1 treatment. The P. aeruginosa (PA) biofilm shows disruption of nearly 50% upon treatment with SPARCL1 at about 0.00134 nM concentration.

FIG. 7D shows the anti-biofilm effects of SPARCL1 at various concentrations. Crystal violet (CV) staining of 3-day old S. typhimurium (ST) biofilm is shown in FIG. 7C in response to nM ranges of SPARCL1 treatment. The S. typhimurium (ST) biofilm shows disruption of nearly 50% upon treatment with SPARCL1 at about 0.00134 nM concentration. Each value is the mean±SEM (n=3, biological replicates). All statistical significance was assessed by using Student's paired t-test, *p<0.05. As demonstrated in FIG. 7A-FIG. 7D, ENO3 and SPARCL1 exhibit strong anti-biofilm activities against multiple microbial strains.

FIG. 8A provides exemplary images of wounded and unwounded healthy de-epidermised dermis human skin equivalent (DED-HSE) models at day 0. The Methylthiazol tetrazolium (MTT) staining of keratinocytes in the DED-HSE samples shows localization of keratinocytes in each sample. As can be seen from the wounded image, there is a lack of keratinocytes in the wounded region at day 0.

FIG. 8B shows the Methylthiazol tetrazolium (MTT) staining of keratinocytes in healthy de-epidermised dermis human skin equivalent (DED-HSE) models at Day 2 and Day 8 before and after wound. The samples are treated with conditioned media obtained from adipose-derived mesenchymal stem cells (ASCs) undergoing adipogenic differentiation at different stages during differentiation (0 day, 6 days, or 12 days). Wounded de-epidermised dermis human skin equivalent (DED-HSE) samples treated with conditioned media obtained from adipose-derived mesenchymal stem cells (ASCs) at 6 days after differentiation (intermediately differentiating) shows concentrated keratinocytes at wounded region, indicating proliferation of keratinocytes at the wounded region.

FIG. 8C provides a quantification of the recovery of the wounded area based on Methylthiazol tetrazolium (MTT) stained images. Data represented as mean±SD. Consistent with the observation in FIG. 8B, the wounded area is covered by keratinocytes in wounded sample treated with conditioned media from intermediately differentiating stem cells.

FIG. 8D provides a hemotoxylin and eosin (H&E) staining revealing a complete wound closure for skin sample treated with Day 6 conditioned media, indicating accelerated wound healing. Dashed lines indicate the wound boundaries. The conditioned media used is from ATCC healthy subject cell line. FIG. 8D demonstrated would closure and recovery of the skin model in samples treated with conditioned media obtained from intermediately differentiating mesenchymal stem cells. Therefore, apart from the anti-bacterial effects, the conditioned media described herein also shows the ability to promote proliferation and wound healing.

FIG. 9 shows the cell confluency of human keratinocytes assessed in a 2D scratch assay at each timepoint by applying an ImageJ plugin PHANTAST on phase contrast time lapse images. The rate of change of confluency was then calculated by obtaining a best fit line across the confluency vs time plot. Each value is the mean±SD (n=2). Conditioned media obtained from intermediately differentiating mesenchymal stem cells and primed with either S. typhimurium (ST) or P. aeruginosa (PA) shows highest migration speed in the scratch assay, supporting the higher wound healing capacity demonstrated in the skin model.

FIG. 10 shows the cell confluency of human keratinocytes assessed in a 2D scratch assay at each timepoint by applying an ImageJ plugin PHANTAST on phase contrast time lapse images. The rate of change of confluency was then calculated by obtaining a best fit line across the confluency vs time plot. Each value is the mean±SD (n=2). The higher keratinocytes migration rate observed in FIG. 9 for the conditioned media can be recapitulated in isolated ENO3 and SPARCL1. Moreover, no cell migration is observed in the Gentamicin treatment group, demonstrating the effects of wound healing for ENO3 and SPARCL1 is independent of anti-bacterial activity from standard antibiotics.

FIG. 11 provides in vivo evidence of anti-bacterial effects of ENO3 and SPARCL1 in a murine excisional wound infection model. Excisional wounds in mice are infected with P. aeruginosa for 24 hours followed by ENO3 or SPARCL1 treatment for 24 hours. Each value is the mean±SEM (n=5). The Colony Forming Units (CFU) counts are evaluated in the treated and untreated sample, indicating the viability of the P. aeruginosa bacteria in each sample. Reduction in bacterial colonies is observed for both ENO3 and SPARCL1 treated samples, indicating anti-bacterial efficacy of both ENO3 and SPARCL1 in vivo.

FIG. 12A shows bacterial viability of P. aeruginosa (PA) treated with short peptide E1A from ENO3 at various concentrations. To develop clinically useful peptides, short peptide regions are identified within ENO3, such as region E1A. The short peptide E1A exhibits more than 50% anti-bacterial activity at the concentration of 0.39 μM.

FIG. 12B shows bacterial viability of P. aeruginosa (PA) treated with short peptide S1A from SPARCL1 at various concentrations. The short peptide S1A exhibits more than 50% anti-bacterial activity at the concentration of 100 μM.

FIG. 12C shows bacterial viability of P. aeruginosa (PA) treated with short peptide S1B from SPARCL1 at various concentrations. The short peptide S1B exhibits more than 50% anti-bacterial activity at the concentration of 50 μM. The P. aeruginosa (PA) bacterial strain used in FIG. 12A-FIG. 12C is grown as per CLSI protocol. Each value is the mean±SD (n=3). All statistical significance was assessed by using Student's paired t-test, *p<0.05. FIG. 12A-FIG. 12C provide in vitro experimental data demonstrating the anti-bacterial activity of short peptides E1A (based on ENO3), S1A (based on SPARCL1), and S1B (based on SPARCL1) against exemplary bacterial strain P. aeruginosa (PA).

FIG. 13A provides Crystal Violet (CV) staining of 3-day old P. aeruginosa (PA) biofilms in response to μM ranges of E1A treatment. The short peptide E1A exhibits more than 50% biofilm disruption activity within the exemplary range from 0.39 μM-1.56 μM.

FIG. 13B provides Crystal Violet (CV) staining of 3-day old P. aeruginosa (PA) biofilms in response to μM ranges of S1A treatment. The short peptide S1A exhibits more than 50% biofilm disruption activity within the exemplary range from 0.78 μM-3.125 μM.

FIG. 13C provides Crystal Violet (CV) staining of 3-day old P. aeruginosa (PA) biofilms in response to μM ranges of S1B treatment. The short peptide S1B exhibits more than 50% biofilm disruption activity at exemplary concentrations from 6.25 μM-12.5 μM. Each value is the mean±SEM (n=3, biological replicates). All statistical significance was assessed by using Student's paired t-test, *p<0.05. FIG. 13A-FIG. 13C provide in vitro experimental data demonstrating the biofilm disruption activity of short peptides E1A (based on ENO3), S1A (based on SPARCL1), and S1B (based on SPARCL1) against exemplary bacterial strain P. aeruginosa (PA).

FIG. 14A further provides Crystal Violet (CV) staining of 3-day old P. aeruginosa (PA) biofilms in response to E1A in combinations with exemplary antibiotic ciprofloxacin. Combined treatment of E1A (concentration: μM) and ciprofloxacin (concentration: μM) shows improved effects in disrupting biofilm formed by exemplary bacterial strain P. aeruginosa (PA), compared to antibiotic alone. The biofilm staining reading for E1A and ciprofloxacin in combination is lower than E1A treatment alone as well, indicating a synergistic effect between the two agents.

FIG. 14B provides Crystal Violet (CV) staining of 3-day old P. aeruginosa (PA) biofilms in response to E1A (concentration: μM) in combinations with exemplary antibiotic colistin (concentration: μM). Each value is the mean±SEM (n=3, biological replicates). Similar to ciprofloxacin, the combined treatment of E1A and colistin shows improved effects in disrupting biofilm formed by exemplary bacterial strain P. aeruginosa (PA), compared to antibiotic alone. The biofilm staining reading for E1A and colistin in combination is comparable to the E1A treatment alone, but at a lower concentration of E1A, indicating a synergistic effect between the two agents.

FIG. 14C provides Crystal Violet (CV) staining of 3-day old P. aeruginosa (PA) biofilms in response to S1A (concentration: μM) in combinations with exemplary antibiotic ciprofloxacin (concentration: μM). Combined treatment of S1A and ciprofloxacin shows improved effects in disrupting biofilm formed by exemplary bacterial strain P. aeruginosa (PA), compared to antibiotic alone. The biofilm staining reading for S1A and ciprofloxacin in combination is lower than S1A treatment alone as well, indicating a synergistic effect between the two agents.

FIG. 14D provides Crystal Violet (CV) staining of 3-day old P. aeruginosa (PA) biofilms in response to S1B (concentration: μM) in combinations with exemplary antibiotic ciprofloxacin (concentration: μM). Combined treatment of S1B and ciprofloxacin shows improved effects in disrupting biofilm formed by exemplary bacterial strain P. aeruginosa (PA), compared to antibiotic alone. The biofilm staining reading for S1B and ciprofloxacin in combination is lower than S1B treatment alone as well, indicating a synergistic effect between the two agents.

FIG. 14E provides Crystal Violet (CV) staining of 3-day old P. aeruginosa (PA) biofilms in response to ENO3 in combinations with exemplary antibiotic ciprofloxacin. Biofilm Control in indicate ‘Media’. All statistical significance was assessed by using Student's paired t-test, *p<0.05. Combined treatment of ENO3 (concentration: nM) and ciprofloxacin (concentration: μM) shows improved effects in disrupting biofilm formed by exemplary bacterial strain P. aeruginosa (PA), compared to antibiotic alone. The biofilm staining reading for ENO3 and ciprofloxacin in combination is lower than ENO3 treatment alone as well, indicating a synergistic effect between the two agents.

FIG. 14F provides Crystal Violet (CV) staining of 3-day old P. aeruginosa (PA) biofilms in response to SPARCL1 in combinations with exemplary antibiotic ciprofloxacin as indicated. Biofilm Control in indicate ‘Media’. All statistical significance was assessed by using Student's paired t-test, *p<0.05. Combined treatment of SPARCL1 (concentration: nM) and ciprofloxacin (concentration: μM) shows improved effects in disrupting biofilm formed by exemplary bacterial strain P. aeruginosa (PA), compared to antibiotic alone. The biofilm staining reading for SPARCL1 and ciprofloxacin in combination is lower than SPARCL1 treatment alone as well, indicating a synergistic effect between the two agents.

FIG. 15 shows cell confluency of human keratinocytes assessed in a 2D scratch assay at each timepoint by applying an ImageJ plugin PHANTAST on phase contrast time lapse images. The rate of change of confluency is calculated by obtaining a best fit line across the confluency vs time plot. Each value is the mean±SD (n=3). The higher migration speed for E1A treated cells compared to media indicates higher wound healing capacity for E1A treated cells.

FIG. 16 provides a summary of an exemplary workflow of preparing the conditioned medium from adipose tissue and identifying novel anti-bacterial peptides. The conditioned medium obtained demonstrate anti-bacterial, anti-biofilm, and wound healing effects, which are effective against pathogens with antibiotic resistance.

### DEFINITIONS

As used herein, the term “chronic wounds” refers to a wound that cannot achieve anatomical and functional integrities through normal, orderly, and timely repair processes under the influence of various internal or external factors. Wound healing, as a normal biological process in the human body, is achieved through four precisely and highly programmed phases: hemostasis, inflammation, proliferation, and remodeling. For a wound to heal successfully, all four phases must occur in the proper sequence and time frame. Chronic wounds are injuries that have not healed and have no tendency to heal after more than an extended period, for example, one month of treatment. Examples of chronic wounds include, nonhealing or infected surgical or traumatic wounds, venous ulcers, pressure ulcers, diabetic foot ulcers, and ischemic ulcers.

As used herein, the term “nosocomial infection” or “healthcare-associated infections (HAI)” refers to an infection that are not present or incubating at the time of admission to a treatment and occurs during or after treatment. These infections include catheter-associated urinary tract infections, central line-associated bloodstream infections, surgical site infections, ventilator-associated pneumonia, hospital-acquired pneumonia, and Clostridium difficile infections.

As used herein, the term “diabetes mellitus”, or “diabetes” refers to a heterogeneous complex metabolic disorder characterized by hyperglycemia, i.e. elevated blood glucose concentration secondary to either resistance to the action of insulin, insufficient insulin secretion, or both. Commonly, diabetes can be classified into two categories, the type 1 and type 2 diabetes. Type 1 diabetes is characterized by an absolute deficiency of insulin secretion. Individuals at increased risk of developing this type of diabetes can often be identified by serological evidence of an autoimmune pathologic process occurring in the pancreatic islets and by genetic markers. For type 2 diabetes, the cause is a combination of resistance to insulin action and an inadequate compensatory insulin secretory response. Statistically, most patients with type 2 diabetes are obese, and obesity itself causes some degree of insulin resistance. Diabetic patients are in particular susceptible to infections, which accompany chronic hyperglycemia.

As used herein, the term “diabetic foot ulcer (DFU)” or “diabetic foot infections (DFI)” is one of the most common and challenging complication associated with the onset of diabetes mellitus, which lowers the quality of life and results in significant morbidity and mortality for patients. In diabetic patients, the chronic foot ulcer is typically caused by poor blood circulation and peripheral neuropathy, which is localized nerve damage. Treatment for diabetic foot ulcer (DFU) is particularly challenging due to the high recurrence rate and antibiotic resistance.

As used herein “anti-microbial resistance (AMR)” or “drug resistance” refers to the ability for pathogens to evade the killing of anti-microbials. Pathogens with anti-microbial resistance (AMR) no longer responds to medicines, thus making infections harder to treat and increasing the risk of disease spread, severe illness and death. Anti-microbials includes, and are not limited to antibiotics, antivirals, antifungals and antiparasitics. Anti-microbials are medicines used to prevent and treat infections in humans, animals and plants.

As used herein, the term “multidrug resistant (MDR) pathogens” refers to pathogens that have become resistant to certain antibiotics, and these antibiotics can no longer be used to control or kill the pathogen. Such pathogen includes, but are not limited to fungi, protists, or bacteria. Exemplary multidrug-resistant fungi include Candida auris, which is resistant to multiple antifungal drugs commonly used to treat Candida infections. Some strains are resistant to all three available classes of antifungals drugs. Multidrug resistant bacteria are commonly found in healthcare facilities, elderly care centers, for examples, and include bacteria strains such as methicillin-resistant Staphylococcus aureus (MRSA), vancomycin-resistant Enterococci (VRE), extended-spectrum β-lactamase (ESBLs) producing Gram-negative bacteria, and Klebsiella pneumoniae carbapenemase (KPC) producing Gram-negative bacteria.

As used herein, the term “ESKAPE pathogen” is an acronym comprising the scientific names of six highly virulent and antibiotic resistant bacterial pathogens, which are Enterococcus faecium, Staphylococcus aureus, Klebsiella pneumoniae, Acinetobacter baumannii, Pseudomonas aeruginosa, and Enterobacter spp. These bacterial pathogens can evade or ‘escape’ commonly used antibiotics due to their increasing multi-drug resistance (MDR).

As used herein, the term “angiogenesis” refers to the growth of blood vessels. The term “angiogenesis-inducing growth factors” refers to growth factors that induce the growth of blood vessels, such as vascular endothelial growth factor (VEGF) or basic fibroblast growth factor (bFGF).

As used herein, the term “mesenchymal stem cells (MSCs)”, or “mesenchymal stromal cells” are multipotent stem cells found in bone marrow that are important for making and repairing skeletal tissues, such as cartilage, bone and the fat found in bone marrow. Mesenchymal stem cells are able to differentiate into a variety of cell types, such as osteoblasts, chondrocytes, myocytes, and adipocytes. With age and disease, mesenchymal stem cells predominantly convert into lipid-accumulating fat cells.

As used herein, the term “adipose-derived mesenchymal stem cells (ASCs)” refers to mesenchymal stem cells (MSCs) that are obtained from adipose tissue. While two types of adipose tissue exist (brown and white), white adipose yields the commonly studied adipose-derived stem cells (ASCs). Methods of obtaining adipose-derived mesenchymal stem cells (ASCs) are known in the art. Adipose tissues are minced and then undergo enzymatic digestion with collagenase type II. After centrifugation, the resulting pellet is called the stroma vascular fraction (SVF). Approximately 2 to 6 million cells in stroma vascular fraction (SVF) can be obtained from one milliliter of lipoaspirate. Stroma vascular fraction (SVF) contains adipose-derived mesenchymal stem cells (ASCs), endothelial cells, endothelial progenitor cells, pericytes, smooth muscle cells, leukocytes, and erythrocytes. Adipose-derived mesenchymal stem cells (ASCs) are obtained as the plastic-adherent population after overnight culturing.

As used herein, the term “intermediately differentiating cells” or “intermediately differentiating adipose-derived mesenchymal stem cells (ASCs)” refers to tissue-derived stem cells or specifically, adipose-derived mesenchymal stem cells (ASCs), that are midway through the process of adipogenic differentiation in vitro and have not yet completed the differentiation into adipocytes (i.e. non-fully differentiated adipocytes). Adipogenic differentiation timeline varies based on the species. For example, human adipose-derived mesenchymal stem cells (ASCs) adipogenic differentiation takes about 12-14 days; mice adipogenic differentiation takes about 8-10 days; and fish adipogenic differentiation takes about 3-6 days. Thus, to generate intermediate stem cells undergoing mid-adipogenesis, the timeline would depend on the species under investigation. As used herein, the intermediately differentiating adipose-derived mesenchymal stem cells (ASCs) from human refers to adipose-derived mesenchymal stem cells (ASCs) that are subjected to adipogenic differentiation for about 4 days to 8 days or about 4 days, about 5 days, about 6 days, about 7 days, or about 8 days.

As used herein, the term “biofilm” refers to a membranous tissue formed by pathogens such as bacteria attached to a chronic wound bed and fused with extracellular matrix secreted by the film. It is composed of pathogens and their products, extracellular matrix, necrotic tissue, and so on. Clinically, biofilm is more common in pressure ulcer, diabetic foot ulcer, lower extremity arteriovenous ulcer, and other chronic wounds.

As used herein, the term “minimum inhibitory concentration (MIC)” measures the in vitro levels of susceptibility or resistance of specific bacterial strains to applied antibiotic. Minimum inhibitory concentration (MIC) is the lowest concentration of an antibacterial agent expressed in mg/L (μg/mL) which, under strictly controlled in vitro conditions, completely prevents >99% of visible growth of the test strain of an organism. As used herein, the term “minimum bactericidal concentration (MBC)” measures the lowest concentration of antibacterial agent required to kill >99% of a particular bacterium. As used herein, the term “minimum biofilm inhibitory concentration (MBIC)” is defined as the lowest concentration of the anti-bacterial agent to inhibit the initial formation of biofilm. At this concentration, there is no time-dependent increase in the average number of viable cells in the biofilm.

The expression “microbe” or “microorganism” as used herein refers to bacteria, protozoa, algae, and fungi.

As used herein, the expression “anti-bacterial response” refers to any host process triggered by the exposure to a bacterium that results in a change in state, activity or viability of the microorganism—for example, movement, secretion, enzyme production or gene expression. For example, an anti-bacterial response elicited by the adipose-derived mesenchymal stem cells (ASCs) as described herein after bacterial priming step includes the production of anti-bacterial peptides into the culturing media.

### DETAILED DESCRIPTION OF THE PRESENT INVENTION

Chronic wound management is a large and growing problem in healthcare. As of today, antibiotics stand as the first line of treatment against wound infections. However, due to the drug resistance in most chronic wound causing pathogens, standard antibiotics treatments show little effect. The emergence of antibiotic resistant infections accompanied with the dearth of new antibiotics possess the biggest challenge. Therefore, what is needed is novel anti-microbial agent effective in treating chronic infections. In one example, the anti-microbial agent can be effective in treating chronic infections, in particular those caused by anti-microbial resistant pathogens.

Mesenchymal stem cells (MSCs) are attractive alternative cell-based therapy to chronic wound treatment due to advantages of easy isolation, relative abundance, anti-inflammatory effects, angiogenic modulation, and homing capacities. Mesenchymal stem cells (MSCs) have been investigated in a wide spectrum of disease indications. Mesenchymal stem cells (MSCs) have been shown to upregulate antibacterial activities by secretion of antibacterial peptides such as LL-37, cathelicidin, lipocalin-2, β-defensin-2, hepcidin and elafin. Standing ahead of existing methods and know peptides, the present disclosure investigated the anti-bacterial function of adipogenic differentiating mesenchymal stem cells (MSCs) based on bacterial priming strategy and isolated novel anti-bacterial peptides useful for the treatment of chronic infections.

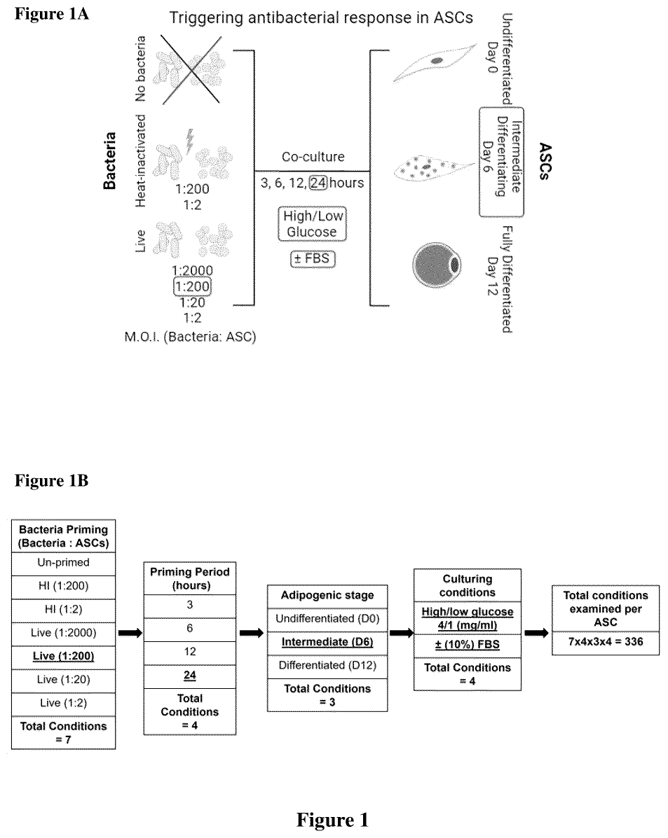

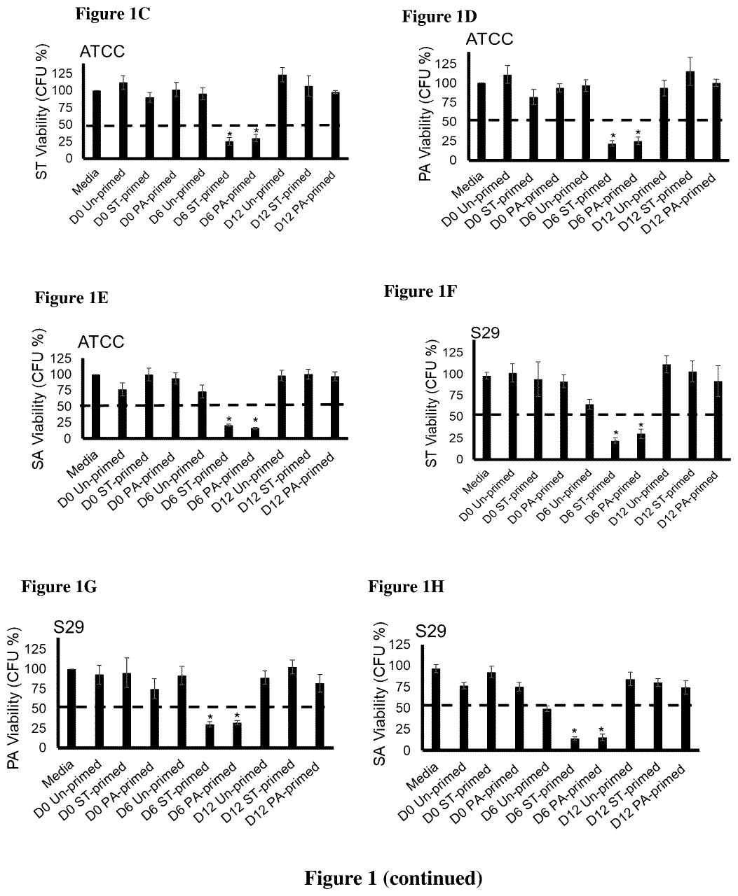

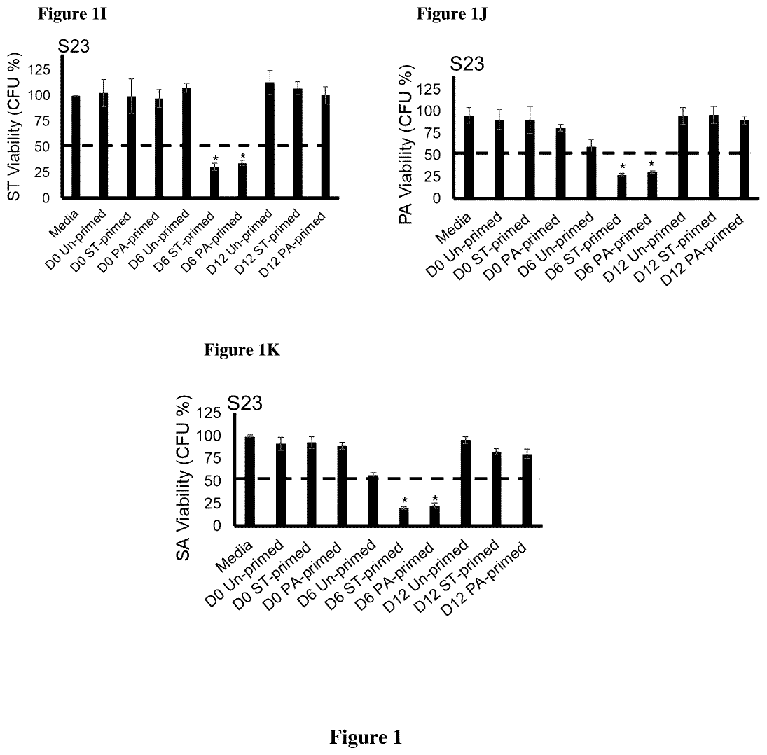

In one aspect, the present disclosure provides anti-bacterial peptides. The anti-bacterial peptides as disclosed herein are isolated from conditioned media obtained from intermediately differentiating mesenchymal stem cells primed with bacterial strains. FIG. 1A provides a summary of parameters in obtaining the conditioned media. Cell viability tests and biofilm staining of exemplary pathogenic bacteria demonstrated the anti-bacterial ability of the identified peptides, as detailed in the experimental section. In one example, the anti-bacterial peptide has or comprises the amino acid sequence of KVNQIGSVTESIQACKLAQSN (E1A, SEQ ID NO: 3). In another example, the anti-bacterial peptide has or comprises the amino acid sequence of HHKAEKSSVLKSKEESH (S1A, SEQ ID NO: 4). In another example, the anti-bacterial peptide has or comprises the amino acid sequence of KQRNKVKKIYLDEKR (S1B, SEQ ID NO: 5). In some examples, the anti-bacterial peptide is at least about 15 amino acids in length.

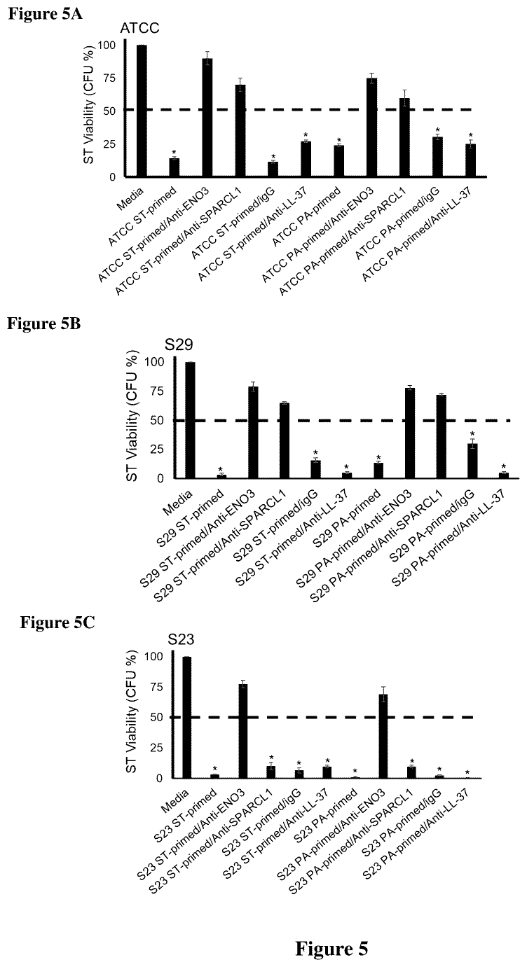

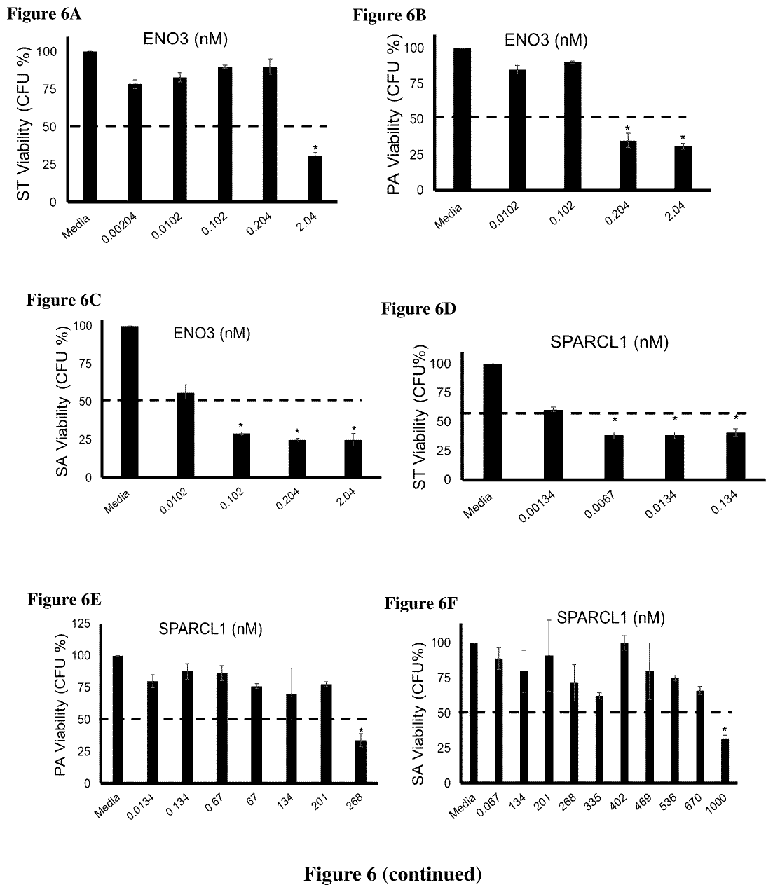

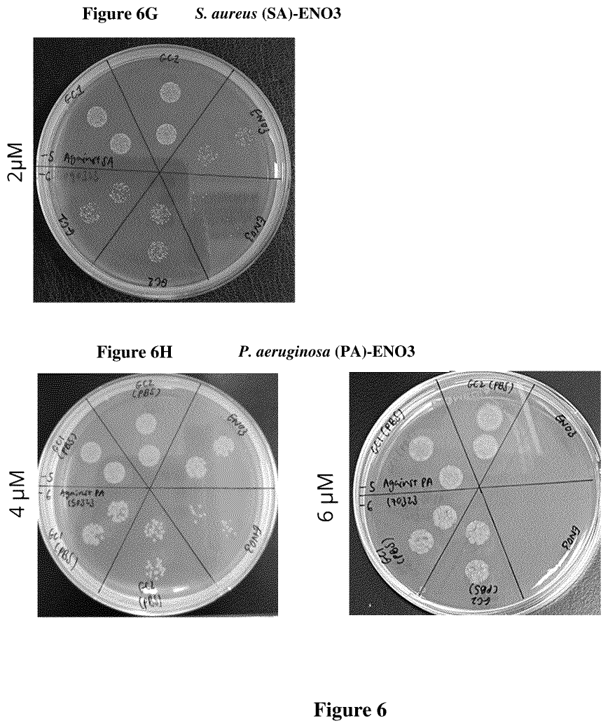

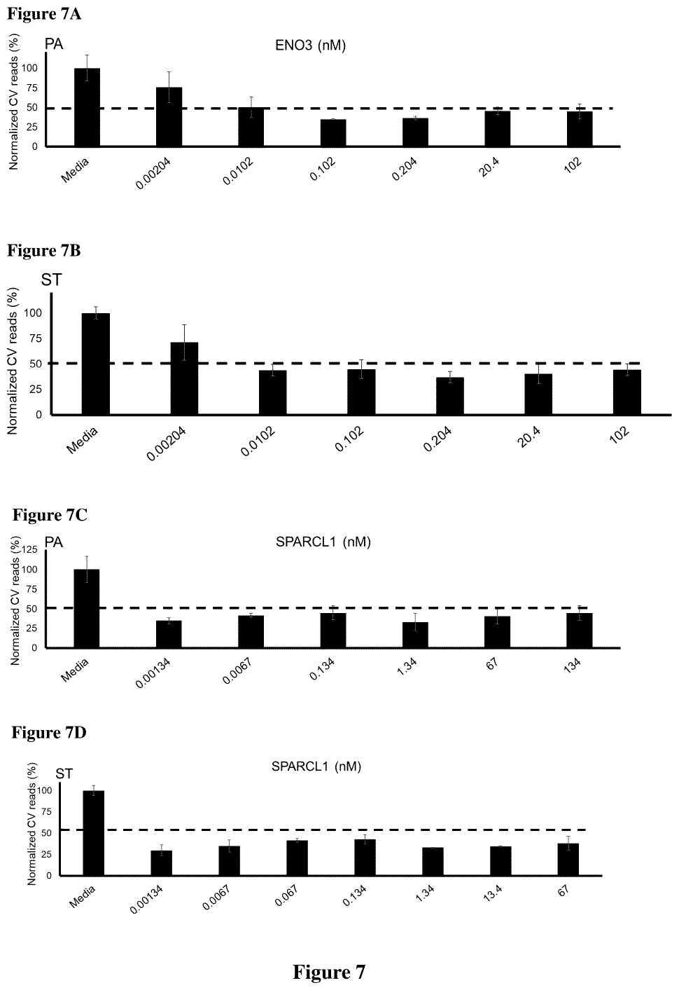

In some examples, the anti-bacterial peptide comprises one or more partial sequence of ENO3, wherein the anti-bacterial peptide comprises at least the sequence of KVNQIGSVTESIQACKLAQSN (SEQ ID NO: 3). That is, the peptide sequence can comprise a partial sequence of ENO3 and the peptide sequences of SEQ ID NO: 3. The partial sequence of ENO3 can be from any region of ENO3, and of any length. In some cases, the partial sequence may be at the N-terminus of SEQ ID NO: 3, or at the C-terminus of SEQ ID NO: 3, or both. As demonstrated in FIG. 5A-5C and FIG. 6A-6F, ENO3 and SPARCL1 show anti-bacterial activity at nM ranges against exemplary bacterial strains. FIG. 7A-FIG. 7D further exemplified the biofilm disruption ability of ENO3 and SPARCL1. In one example, the full-length amino acid sequence of human ENO3 is SEQ ID NO: 1. In some examples, the anti-bacterial peptide comprises a partial sequence of SPARCL1, wherein the anti-bacterial peptide comprises at least the sequence of HHKAEKSSVLKSKEESH (SEQ ID NO: 4). That is, the peptide sequence can comprise a partial sequence of SPARCL1 and the peptide sequences of SEQ ID NO: 4 or 5. The partial sequence of SPARCL1 can be from any region of SPARCL1, and of any length. In some cases, the partial sequence may be at the N-terminus of SEQ ID NO: 4 or 5, or at the C-terminus of SEQ ID NO: 4 or 5, or both. In some examples, the anti-bacterial peptide comprises a partial sequence of SPARCL1, wherein the anti-bacterial peptide comprises at least the sequence of KQRNKVKKIYLDEKR (SEQ ID NO: 5). In one example, the full-length amino acid sequence of human SPARCL1 is SEQ ID NO: 2. Sequences for full-length human ENO3 and full-length human SPARCL1 are disclosed in Table 2. In some other examples, the full-length ENO3 or SPARCL1 sequences can be from other species, for example, from a mammal such as mice, monkey, rabbit, dog, or hamster. In some other examples, the full-length ENO3 or SPARCL1 sequences can be from a fish.

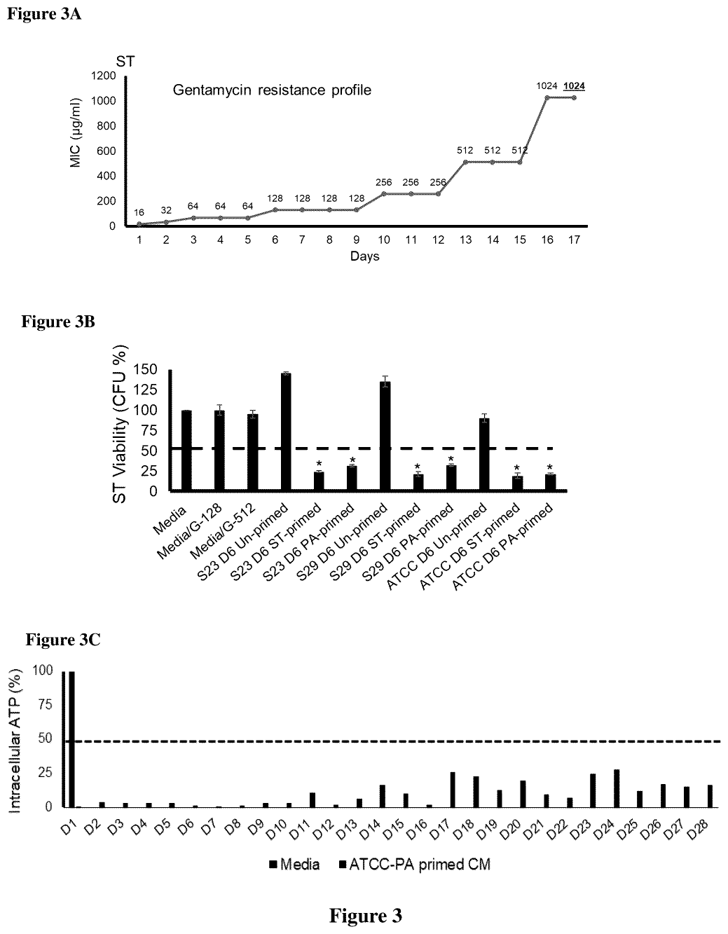

In another aspect, the present disclosure provides a conditioned cell culture media (CM). In one example, the conditioned cell culture media (CM) comprises the anti-bacterial peptides as described herein. In another example, the conditioned cell culture media (CM) comprises a full length ENO3 peptide, or a full length SPARCL1 peptide as described herein. In some examples, the media is non-cytotoxic. The conditioned cell culture media is obtained based on the exemplary conditions as shown in FIG. 1A and FIG. 1B. In one example, the conditioned cell culture media (CM) is effective against microbes with multidrug resistance (MDR). As shown in FIG. 3B and FIG. 3C, for example, the viability of drug-resistant bacterial reduces to less than 50% after treatment with the conditioned media as described herein.

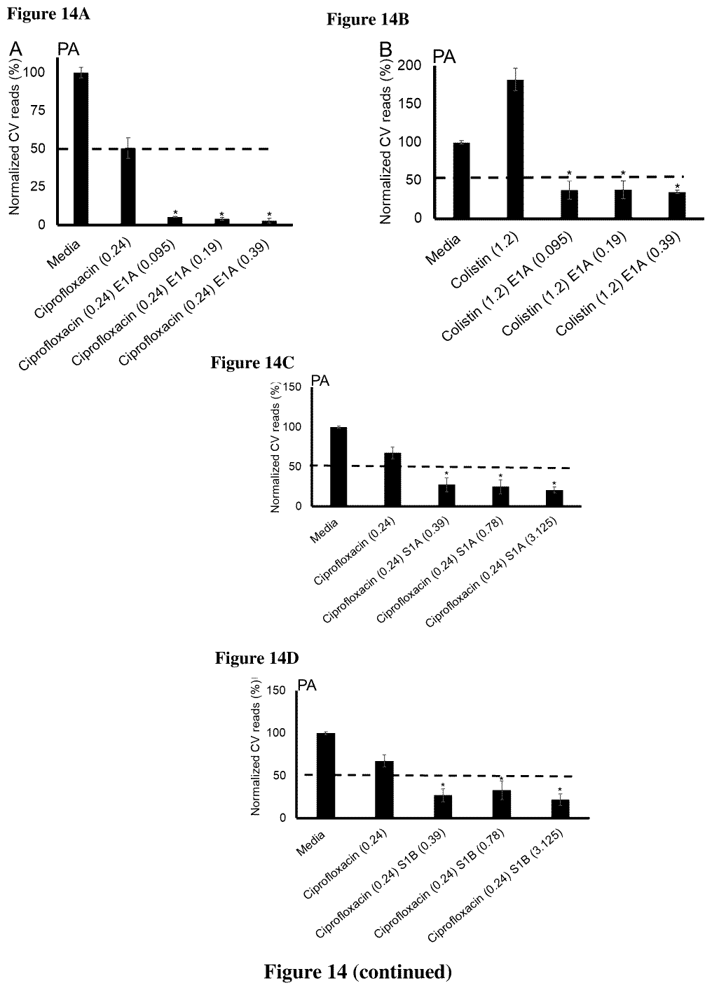

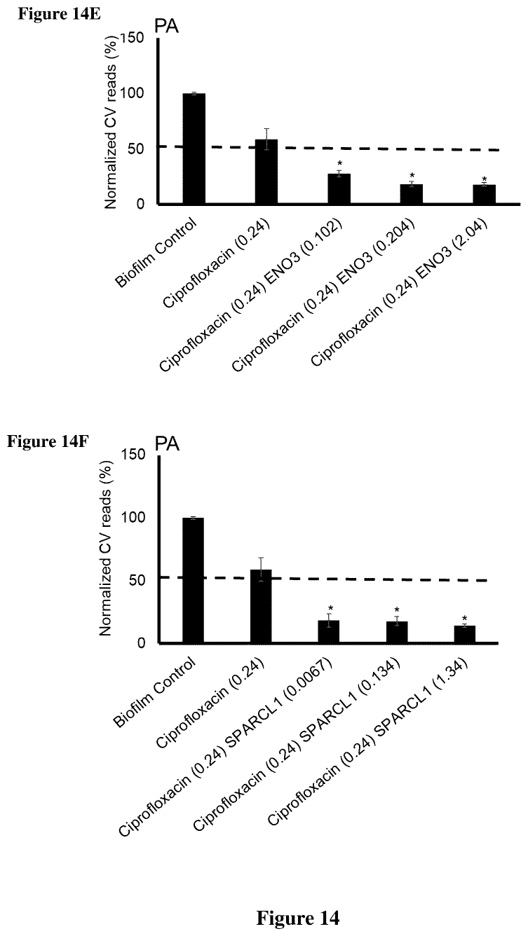

In another aspect, the present disclosure provides a composition comprising one or more of the anti-bacterial peptides as described herein. In one example, the composition comprises a full length ENO3 peptide, or a full length SPARCL1 peptide as described herein. In another example, the composition further comprises an antibiotic agent. In one example, wherein the composition comprises a full length ENO3 peptide, or a full length SPARCL1 peptide as described herein, the composition also comprises an antibiotic agent. FIG. 14E and FIG. 14F shows the biofilm disrupting effects in combination with exemplary antibiotic ciprofloxacin. Other antibiotics comprised in the composition can be, and are not limited to, gentamycin ((3R,4R,5R)-2-{[(1S,2S,3R,4S,6R)-4,6-diamino-3-{[(2R,3R,6S)-3-amino-6-[(1R)-1-(methylamino)ethyl]oxan-2-yl]oxy}-2-hydroxycyclohexyl]oxy}-5-methyl-4-(methylamino)oxane-3,5-diol), colistin (N-(4-amino-1-(1-(4-amino-1-oxo-1-(3,12,23-tris(2-aminoethyl)-20-(1-hydroxyethyl)-6,9-diisobutyl-2,5,8,11,14,19,22-heptaoxo-1,4,7,10,13,18-hexaazacyclotricosan-15-ylamino)butan-2-ylamino)-3-hydroxybutan-2-ylamino)-1-oxobutan-2-yl)-N,5-dimethylheptanamide) piperacillin ((2S,5R,6R)-6-{[(2R)-2-[(4-ethyl-2,3-dioxo-piperazine-1-carbonyl)amino]-2-phenyl-acetyl]amino}-3,3-dimethyl-7-oxo-4-thia-1-azabicyclo[3.2.0]heptane-2-carboxylic acid), kanamycin A (2-(aminomethyl)-6-[4,6-diamino-3-[4-amino-3,5-dihydroxy-6-(hydroxymethyl) tetrahydropyran-2-yl]oxy-2-hydroxy-cyclohexoxy]-tetrahydropyran-3,4,5-triol), kanamycin B ((2S,3R,4S,5S,6R)-4-amino-2-{[(2S,3R,4S,6R)-4,6-diamino-3-{[(2R,3R,4R,5S,6R)-3-amino-6-(aminomethyl)-4,5-dihydroxyoxan-2-yl]oxy}-2-hydroxycyclohexyl]oxy}-6-(hydroxymethyl)oxane-3,5-diol), nalidixic acid (1-Ethyl-7-methyl-4-oxo-[1,8]naphthyridine-3-carboxylic acid), ampicillin ((2S,5R,6R)-6-([(2R)-2-Amino-2-phenylacetyl]amino)-3,3-dimethyl-7-oxo-4-thia-1-azabicyclo[3.2.0]heptane-2-carboxylic acid), vancomycin ((1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy}-22-(carbamoylmethyl)-5,15-dichloro-2,18,32,35,37-pentahydroxy-19-[(2R)-4-methyl-2-(methylamino)pentanamido]-20,23,26,42,44-pentaoxo-7,13-dioxa-21,24,27,41,43-pentaazaoctacyclo[26.14.2.2^{3,6}.2^{14,17}.1^{8,12}.1^{29,33}.0^{10,25}.0^{34,39}]pentaconta-3,5,8(48),9,11,14,16,29(45),30,32,34,36,38,46,49-pentadecaene-40-carboxylic acid), linezolid ((S)—N-({3-[3-fluoro-4-(morpholin-4-yl)phenyl]-2-oxo-1,3-oxazolidin-5-yl}methyl)acetamide) and ciprofloxacin (1-cyclopropyl-6-fluoro-4-oxo-7-(piperazin-1-yl)-quinoline-3-carboxylic acid).

In another aspect, the present disclosure provides a pharmaceutical composition comprising the peptide as described herein. In another example, the present disclosure provides a pharmaceutical composition comprising the conditioned cell culture media as described herein. In a further example, the present disclosure provides a pharmaceutical composition comprising the composition as disclosed herein. In some examples, the pharmaceutical composition further comprises a pharmaceutically acceptable carrier and/or excipient. A person skilled in the art would be able to provide suitable carrier or excipients for the manufacture of pharmaceutical composition based on the dosage form or routes of administration needed.

In a further aspect, the present disclosure provides a wound dressing comprising the peptide as described herein, the conditioned cell culture media (CM) as described herein, the composition as described herein, or the pharmaceutical composition as described herein. As used herein, the term “wound dressing” refers to a sterile pad applied to a wound to promote healing and protect the wound from further harm. A dressing is designed to be in direct contact with the wound, which may or may not be self-adhesive. In some cases, a wound dressing may be medicated, i.e. comprising one or more medicaments suitable for wound healing, suppression of infection, pain management, or other purposes. Material of a wound dressing differs based on the wound type. For example, the wound dressing can be, and is not limited to an alginate dressing, a hydrocolloid dressing, or a foam dressing. A person skilled in the art would be able to assess the type, shape, and position of the wound to be treated and select suitable material and type of dressing to be used. The term “conditioned media” refers to the cell secretome in the form of cell culture media, which comprises a collection of proteins shed from cell surface and intracellular proteins released through non-classical secretion pathway or exosomes. These secreted proteins include enzymes, growth factors, cytokines, hormones, and/or other soluble mediators. As used herein, the term “conditioned media” refers to the cell culture media after culturing of the tissue-derived stem cells as described herein. The cell culture media used in the preparation of conditioned media can be cell culture media suitable for culturing of tissue-derived stem cells. Such cell culture media and method of preparing them are known in the art, for example, mesenchymal stem cell growth medium. The medium can be xeno-free or serum-free. In one example, the cell culture medium is an Essential E6 cell culture medium. A person skilled in the art would be able to elect suitable cell culture media based on the type of cells to be cultured, with suitable supplements, if needed.

In another aspect, the present disclosure provides an in vitro method of inducing anti-bacterial responses in tissue-derived stem cells. As used herein, the term “anti-bacterial response” refers to a host process triggered by the exposure to a microorganism that results in a change in state, activity or viability of the microorganism. As used herein, for example, the anti-bacterial response elicited by the mesenchymal stem cells as described herein after bacterial priming includes the production of anti-bacterial peptides. In one example, the anti-bacterial responses produce the anti-bacterial peptides as described herein.

As used herein, the term “tissue-derived stem cell” refers to stem cells derived from somatic tissues which can be differentiated into mesenchymal lineages such as bone, cartilage, fat, and skin. In one example, the tissued-derived stem cell is a mesenchymal stem cell (MSC). In another example, the tissued-derived stem cells can be, but are not limited to: adipose-derived mesenchymal stem cells (ASCs), bone marrow-derived MSCs (BM-MSCs), dental pulp-stem cells, umbilical cord derived MSCs. As used herein, the term “adipose-derived mesenchymal stem cells (ASCs)” refers to mesenchymal stem cells (MSCs) that are obtained from abundant adipose tissue. Adipose-derived mesenchymal stem cells (ASCs) are adherent on plastic culture flasks and can be expanded in vitro. Adipose-derived mesenchymal stem cells (ASCs) have the capacity to differentiate into multiple cell lineages. The exemplary method of inducing antibacterial potency in adipose-derived mesenchymal stem cells (ASCs) as described in the experimental section can be extended to other types of stem cells commonly recruited at the wound site such as epidermal stem cells (EPSCs), hair follicle stem cells (HFSCs), hematopoietic stem cells (HSCs) and other multipotent MSCs.

In one example, present disclosure provides an in vitro method of inducing anti-bacterial responses in tissue-derived stem cells, the method comprises obtaining a tissue sample from a subject. The tissue sample can be, but is not limited to: an adipose tissue, a bone marrow tissue, a dental pulp, or an umbilical cord blood. A person skilled in the art would be able to select suitable type of samples to be obtained from a subject based on the type of tissue-derived stem cell needed. In another example, the subject can be a healthy subject. In a further example, the subject can be an obese subject, or a diabetic subject.

In another example, the present disclosure provides an in vitro method of inducing anti-bacterial responses in tissue-derived stem cells, wherein the method comprises obtaining a tissue sample from a subject and isolating tissue-derived stem cells from the tissue sample in a cell culture. Methods of isolating tissue-derived stem cells are known in the art. For example, to isolate adipose-derived mesenchymal stem cells (ASCs) from fat tissue, based on the widely accepted method disclosed in Zuk P A, Zhu M, Mizuno H, Huang J, Futrell J W, Katz A J, Benhaim P, Lorenz H P, Hedrick M H. Multilineage cells from human adipose tissue: implications for cell-based therapies. Tissue Eng. 2001 April; 7(2):211-28), minced adipose tissue are digested and centrifugated to obtain a stroma vascular fraction (SVF), which contains adipose-derived mesenchymal stem cells (ASCs) in the plastic-adherent population after overnight culturing. A person skilled in the art is capable of obtaining suitable isolation protocols based on the type of cells and tissue samples used.

In another example, the present disclosure provides an in vitro method of inducing anti-bacterial responses in tissue-derived stem cells. Briefly, tissue-derived mesenchymal stem cells such as adipose-derived mesenchymal stem cells are isolated from a tissue sample from a subject. The isolated cells are cultured in adipogenic differentiation media to induce adipogenesis. During mid-adipogenesis, before the differentiation is completed, the cells are co-incubated with live bacterial culture or a bacterial surface antigen to induce an anti-bacterial response. The method comprises obtaining a tissue sample from a subject, isolating tissue-derived stem cells from the tissue sample in a cell culture, and culturing the tissue-derived stem cells in a differentiation media to obtain intermediately differentiating cells. In one example, the differentiation media is adipogenic differentiation media. Various methods of adipogenesis are known in the art. For example, an adipogenesis method is characterized in subjecting the tissue-derived stem cells to the treatment of an adipogenic cocktail comprising indomethacin, insulin, dexamethasone, and IBMX.

In another example, the present disclosure provides an in vitro method of inducing anti-bacterial responses in tissue-derived stem cells, wherein the method comprises obtaining a tissue sample from a subject, isolating tissue-derived stem cells from the tissue sample in a cell culture, culturing the tissue-derived stem cells in a differentiation media to obtain intermediately differentiating cells, and incubating the intermediately differentiating cells with a bacteria culture or a bacterial surface antigen to induce an anti-bacterial response of the intermediately differentiating cells. As used herein, the “intermediately differentiating cells” refers to the tissue-derived stem cells that are undergoing adipogenesis. In one example, the “intermediately differentiating cells” refers to tissue-derived stem cells that are mid-way through adipogenesis. Adipogenic differentiation timeline varies based on the species. For example, human adipose-derived mesenchymal stem cells (ASCs) adipogenic differentiation takes about 12-14 days; mice adipogenic differentiation takes about 8-10 days; and fish adipogenic differentiation takes about 3-6 days. Thus, to generate intermediate stem cells undergoing mid-adipogenesis, the timeline would depend on the species under investigation. As used herein, the intermediately differentiating adipose-derived mesenchymal stem cells (ASCs) from human refers to adipose-derived mesenchymal stem cells (ASCs) that are subjected to adipogenic differentiation for about 4 to 8 days or about 4 days, about 5 days, about 6 days, about 7 days, or about 8 days. In one example, the intermediately differentiating adipose-derived mesenchymal stem cells (ASCs) from mice can be obtained between 2-6 days after adipogenic differentiation. In another example, the intermediately differentiating adipose-derived mesenchymal stem cells (ASCs) from fish can be obtained between 1-4 days after adipogenic differentiation. In one example, the bacterial culture is a culture of an ESKAPE pathogen. In a further example, the bacterium can be, but is not limited to Salmonella typhimurium and Pseudomonas aeruginosa. In another example, the bacterial surface antigen refers to surface proteins, lipopolysaccharides, and peptidoglycans on the bacterial cell wall. The bacterial surface antigens used to induce an anti-bacterial response of the intermediately differentiating cells can be, and are not limited to lipopolysaccharides (LPS), lipoteichoic acid (LTA), and peptidoglycan (PG).

In another aspect, the present disclosure provides an in vitro method of inducing anti-bacterial responses in adipose-derived mesenchymal stem cells (ASCs), comprising: (a) obtaining an adipose tissue sample from a subject; (b) isolating adipose-derived mesenchymal stem cells (ASCs) from the adipose tissue sample of (a) in a cell culture; (c) culturing the adipose-derived mesenchymal stem cells (ASCs) in adipogenic differentiation media to obtain intermediately differentiating ASCs; and (d) incubating the intermediately differentiating ASCs with a bacteria culture or a bacterial surface antigen to induce an anti-bacterial response by the intermediately differentiating ASCs. Complementary to their roles in wound regeneration and regulation of immune responses, adipose-derived mesenchymal stem cells (ASCs) are known for interactions with pathogenic bacteria and bacterial components at the injury site. However, as exemplified in FIG. 1C-FIG. 1K, intermediately differentiating adipose-derived mesenchymal stem cells (ASCs) elicit anti-bacterial response when primed with bacterial culture. In one example of the in vitro method as disclosed herein, the anti-bacterial responses produce the anti-bacterial peptides as described herein.

In another aspect, the present disclosure provides a method of producing the conditioned cell culture media (CM) as described herein, wherein the method comprises (a) obtaining a tissue sample from a subject; (b) isolating tissue-derived stem cells from the tissue sample of (a); (c) culturing the tissue-derived stem cells in a differentiation media to obtain intermediately differentiating stem cells; (d) incubating the intermediately differentiating cells in a cell culture media with a bacteria culture or a bacterial surface antigen; and (e) collecting the conditioned cell culture media (CM) by removing the bacteria and the intermediately differentiating cells. Methods for removing cells from a cell culture are known in the art, for example, by centrifugation at suitable speed to separate the cells and the cell culture media based on their differential weight.

In a further aspect, the present disclosure provides a method of making the conditioned cell culture media (CM) as described herein, wherein the method comprises (a) obtaining an adipose tissue sample from a subject; (b) isolating adipose-derived mesenchymal stem cells (ASCs) from the adipose tissue of (a) in a cell culture; (c) culturing the adipose-derived mesenchymal stem cells (ASCs) in adipogenic differentiation media to obtain intermediately differentiating ASCs; (d) incubating the intermediately differentiating ASCs in a cell culture media with a bacteria culture or a bacterial surface antigen; and (e) collecting the conditioned cell culture media (CM) by removing the bacteria and the intermediately differentiating ASCs.

In one example, the tissue derived stem cell/ASC is a mammalian cell. In some examples, the tissue derived stem cell/ASC can be a human cell, a mice cell, or a fish cell. In some examples, the incubation period for the tissue derived stem cells/ASC in a cell culture media with a bacteria culture or a bacterial surface antigen can be about 14 to 48 hours. In another example, the bacteria in the bacteria culture to be incubated with the intermediately differentiating tissue derived stem cells/ASC is live bacteria. In some further examples, the ratio of the number of bacteria contacting tissue derived stem cells/ASC can be, but is not limited to about 1:50-1:75; about 1:75-1:100, about 1:100-1:200, about 1:200-1:225, and about 1:225-1:250. In some examples, the high or low concentrations of the glucose added, or the presence or absence of additional FBS in the cell culture media do not affect the outcome of the methods as described herein. The specific conditions tested are demonstrated in FIG. 1B. In another example, the subject can be a healthy subject. In a further example, the subject can be an obese subject, or a diabetic subject. Comparison between FIG. 1C-FIG. 1E and FIG. 1F-1H shows that the same conditions are consistently effective in cell lines obtained from healthy subjects or diabetic patients.

In some examples, for the anti-bacterial peptides as described herein, the conditioned cell culture media (CM) as described herein, or the pharmaceutical composition as described herein, the microbial can be a bacterium, a fungus, or a protist. In one example, the fungus is a Candida albicans. In another example, the protists can be, and are not limited to Acanthamoeba spp and Leishmania tropica.

In one aspect, the present disclosure provides a method of treating an infection in a subject. The method comprises administering to the subject a pharmaceutically effective amount of the peptide as described herein, the composition as described herein, or the pharmaceutical composition as described herein. FIGS. 6A-6H demonstrate the effective killing of exemplary microbials by ENO3 and SPARCL1 peptides. FIGS. 12A-12C show the effective killing of exemplary microbials by the short peptides, for example, E1A, S1A, and S1B. As used herein, the term “pharmaceutically effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with the disease state (e.g., relieving the infection). A person skilled in the art is able to determine a pharmaceutically effective amount for the peptide, the composition, or the pharmaceutical composition as disclosed herein based on considerations such as disease state, body size, administration frequencies and route. In some examples, the infection is a mixed infection. In one example, the infection is a bacterial infection. In another example, the subject may further comprise a fungal infection, a protist infection, or a combination thereof. In some examples, the infection can be caused by one or more of the following microorganisms: Salmonella typhimurium, Pseudomonas aeruginosa, Staphylococcus aureus, Enterococcus faecalis, Klebsiella pneumoniae, Actinetobacter baumannii, Escherichia coli, Enterobactor cloacae, Serratia marcescens, Fusobacterium nucleatum, Porphyromonas gingivalis, Prevotella intermedia, Streptococcus sanguinis, or Lactobacillus casei.

In one example, the present disclosure provides the use of the peptide as described herein, the composition as described herein, or the pharmaceutical composition as described herein in the manufacture of a medicament for treating an infection in a subject.

In another example, the infection is a chronic infection, or a nosocomial infection. In a further example, the infection is an infection caused by a drug resistant microorganism. In a further example, the infection is caused by a multi-drug resistant microorganism. In some examples, the infection is resistant to one or more anti-bacterial agents, such as, but are not limited to: vancomycin, ampicillin, gentamicin, kanamycin A, neomycin B, neomycin C, neomycin E, amikacin, tobramycin, dibekacin, sisomicin, netilmicin, streptomycin, plazomicin, ticarcillin, pefloxacin, ceftriaxone, and methicillin. FIGS. 3B and 3C provide an example of a drug resistant microorganism, which respond to the treatment of the exemplary conditioned media described herein even after 28 days of prolonged treatment.

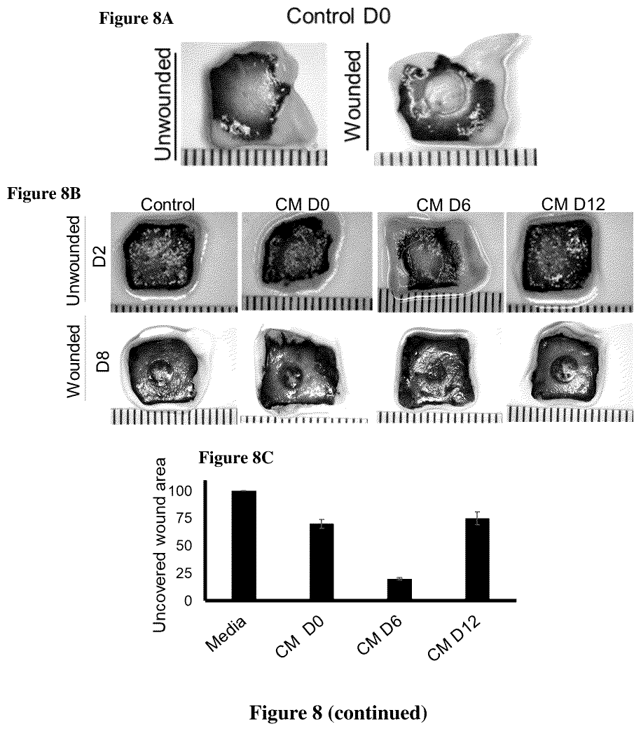

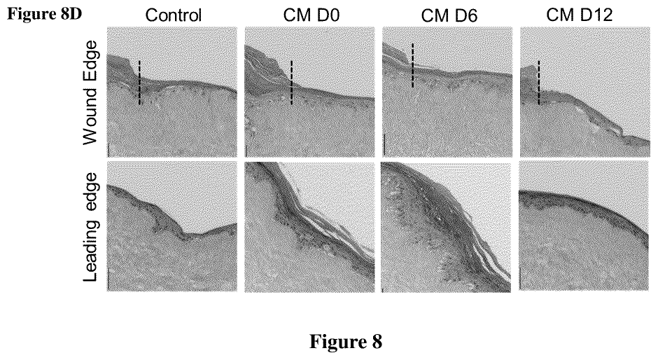

In one aspect, the present disclosure provides a method of promoting wound healing and tissue regeneration in a subject. The method comprises administering to the subject a pharmaceutically effective amount of the peptide as described herein, the composition as described herein, the conditioned media as disclosed herein, or the pharmaceutical composition as described herein. FIGS. 8A-8D provide examples of wounded and unwounded healthy de-epidermised dermis human skin equivalent (DED-HSE) models. Wounded DED-HSE samples treated with conditioned media as described herein shows concentrated keratinocytes at wounded region, indicating proliferation of keratinocytes at the wounded region. FIG. 8D demonstrated would closure and recovery of the skin model in samples treated with conditioned media as described herein. In one example, the wound is not yet showing symptoms of infection. In another example, the wound is an infected wound. In a further example, the wound is suffering from a chronic infection. In one example, the wound is infected by one or more microorganisms. In another example, the wound is infected by one or more bacteria. In some examples, the wound is infected by bacteria including, but are not limited to: Salmonella typhimurium, Pseudomonas aeruginosa, Staphylococcus aureus, Enterococcus faecalis, Klebsiella pneumoniae, Actinetobacter baumannii, Escherichia coli, Enterobactor cloacae, Serratia marcescens, Fusobacterium nucleatum, Porphyromonas gingivalis, Prevotella intermedia, Streptococcus sanguinis, or Lactobacillus casei. In some examples, the bacterium in the wound is resistant to one or more anti-bacterial agents, such as, but are not limited to: vancomycin, methicillin, gentamicin, kanamycin A, amikacin, tobramycin, dibekacin, sisomicin, netilmicin, neomycin B, neomycin C, neomycin E, streptomycin, plazomicin, ticarcillin, pefloxacin, ceftriaxone, and ampicillin.

In one example, the present disclosure provides the use of the peptide as described herein, the composition as described herein, the conditioned media as disclosed herein, or the pharmaceutical composition as described herein in the manufacture of a medicament for promoting wound healing and tissue regeneration in a subject.

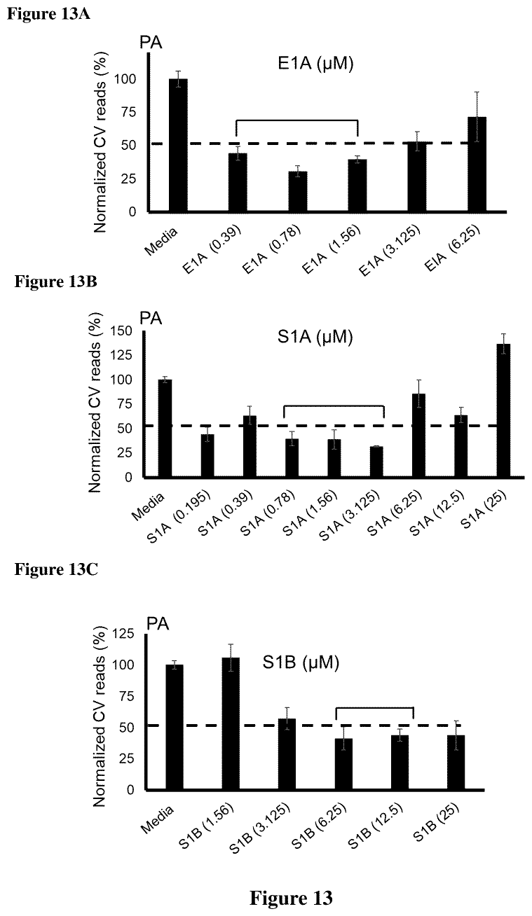

In one aspect, the present disclosure provides a method of reducing/preventing formation of biofilm in a subject. The method comprises administering to the subject a pharmaceutically effective amount of the peptide as described herein, the composition as described herein, the conditioned media as disclosed herein, or the pharmaceutical composition as described herein. FIGS. 7A-7D and FIGS. 13A-13C show effective disruption of the biofilm formed by exemplary microorganisms by the peptides as described herein. In one example, the biofilm is formed by one or more microorganism. In another example, the biofilm comprises one or more bacteria. In a further example, bacteria present in the biofilm can be, and are not limited to: Salmonella typhimurium, Pseudomonas aeruginosa, Staphylococcus aureus, Enterococcus faecalis, Klebsiella pneumoniae, Actinetobacter baumannii, Escherichia coli, Enterobactor cloacae, Serratia marcescens, Fusobacterium nucleatum, Porphyromonas gingivalis, Prevotella intermedia, Streptococcus sanguinis, or Lactobacillus casei. In one example, the bacteria present in the biofilm is resistant to one or more anti-bacterial agents. In a further example, the one or more anti-bacterial agents can be, but are not limited to vancomycin, methicillin, gentamicin, kanamycin A, amikacin, tobramycin, dibekacin, sisomicin, netilmicin, neomycin B, neomycin C, neomycin E, streptomycin, plazomicin, ticarcillin, pefloxacin, ceftriaxone, and ampicillin.

In some examples, the effective biofilm disruption concentration range for E1A can be, but is not limited to at least about 0.1 μM, at least about 0.15 μM, at least about 0.2 μM, at least about 0.25 μM, at least about 0.3 μM, at least about 0.35 μM, or at least about 0.39 μM. In some examples, the effective biofilm disruption concentration range for E1A can be, but is not limited to less than about 3.125 μM, less than about 3 μM, less than about 2.5 μM, less than about 2 μM, less than about 1.75 μM, less than about 1.25 μM, or less than about 1 μM. In one example, the effective biofilm disruption concentration ranges from about 0.39 μM-1.56 μM for E1A.

In some examples, the effective biofilm disruption concentration range for S1A can be, but is not limited to at least about 0.1 μM, at least about 0.2 μM, at least about 0.3 μM, at least about 0.4 μM, at least about 0.5 μM, at least about 0.6 μM, or at least about 0.78 μM. In some examples, the effective biofilm disruption concentration range for S1A can be, but is not limited to less than about 6.25 μM, less than about 6 μM, less than about 5 μM, less than about 4 μM, less than about 4.5 μM, less than about 4.25 μM, less than about 3.75 μM, less than about 3.5 μM, or less than about 3.125 μM. In one example, the effective biofilm disruption concentration ranges from about 0.1 μM-0.3 μM for S1A. In another example, the effective biofilm disruption concentration ranges from about 0.78 μM-3.125 μM for S1A.

In some examples, the effective biofilm disruption concentration range for S1B can be, but is not limited to at least about 3.2 μM, at least about 3.5 μM, at least about 4 μM, at least about 4.5 μM, at least about 5 μM, at least about 5.5 μM, at least about 6 μM, or at least about 6.25 μM. In some examples, the effective biofilm disruption concentration range for S1B can be, but is not limited to less than about 25 μM, less than about 22 μM, less than about 20 μM, less than about 18 μM, less than about 15 μM, less than about 13 μM, or less than about 12.5 μM. In another example, the effective biofilm disruption concentration ranges from about 6.25 μM-12.5 μM for S1B.

In one example, the present disclosure provides the use of the peptide as described herein, the composition as described herein, the conditioned media as disclosed herein, or the pharmaceutical composition as described herein in the manufacture of a medicament for reducing/preventing formation of biofilm in a subject.

In one aspect, the present disclosure provides a method of treating an infection in a subject, wherein the method comprises administering to the subject a pharmaceutically effective amount of the peptide as described herein, a full length ENO3 peptide, or a full length SPARCL1 peptide. In one example, the present disclosure provides the use of the peptide as described herein, a full length ENO3 peptide, or a full length SPARCL1 peptide in the manufacture of a medicament for treating an infection in a subject. In one example, the method further comprises administering to the subject an antibiotic agent. In some examples, the infection to be treated is at a wound, such as a chronic wound. In some examples, the infection to be treated is in an absence of a wound.

In another aspect, the present disclosure provides a method of promoting wound healing and tissue regeneration in a subject, wherein the method comprises administering to the subject a pharmaceutically effective amount of the peptide as described herein, a full length ENO3 peptide, or a full length SPARCL1 peptide. In one example, the present disclosure provides the use of the peptide as described herein, a full length ENO3 peptide, or a full length SPARCL1 peptide in the manufacture of a medicament for promoting wound healing and tissue regeneration in a subject.

In another aspect, the present disclosure provides a method of reducing/preventing formation of biofilm in a subject, wherein the method comprises administering to the subject a pharmaceutically effective amount of the peptide as described herein, a full length ENO3 peptide, or a full length SPARCL1 peptide. In one example, the present disclosure provides the use of the peptide as described herein, a full length ENO3 peptide, or a full length SPARCL1 peptide in the manufacture of a medicament for reducing/preventing formation of biofilm in a subject. In one example, the method further comprises administering to the subject an antibiotic agent.

In one example of the methods as described herein, the peptide and the antibiotic agent are administered together, or separately. The administration can be, and are not limited to intramuscular, topical and intravenous administration. In some examples, the peptides, the conditioned media, the composition, or the pharmaceutical composition as described herein are to be delivered to the subject embedded in a hydrogel, a polymer, or a nanofiber, or attached to a nanoparticle. Delivery systems such as hydrogels or poly(lactic-co-glycolic) acid (PLGA) nanoparticles can increase the bioavailability and stability of the peptides. In some examples, the subject is diagnosed to have, or diagnosed to be at risk of having obesity or diabetes. In some examples, the subject has diabetic foot ulcer and/or diabetic foot infections.

In one aspect, the present disclosure provides a nucleic acid composition encoding the peptide as described herein. In another aspect, the present disclosure provides an expression vector comprising the nucleic acid composition as described herein. In another aspect, the present disclosure provides a host cell comprising one or more of the nucleic acid compositions as described herein, or one or more of the expression vectors as described herein. In one example, the host cell is a mammalian cell, a bacterium, a yeast cell, a fungus cell, or a plant cell. In another example of the methods as disclosed herein, the tissue sample is a fat sample. In some further examples, the tissue sample is a subcutaneous fat sample.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It should further be appreciated that the exemplary embodiments are only examples, and are not intended to limit the scope, applicability, dimensions, or configuration of the invention in any way. Rather, the foregoing detailed description will provide those skilled in the art with a convenient road map for implementing an exemplary embodiment of the invention, it being understood that various changes may be made in the function and arrangement of elements and method of fabrication described in an exemplary embodiment without departing from the scope of the invention as set forth in the appended claims.

### EXPERIMENTAL SECTION

### Adipose-Derived Mesenchymal Stem Cell (ASC) Lines

White adipose tissue (WAT) was isolated from the subcutaneous (abdominal region) depot from all the human volunteers undergoing bariatric surgery with approval from the Domain Specific Review Board at National Healthcare Group, Singapore. Adipose-derived mesenchymal stem cells (ASCs) were isolated and enriched by serial passage culture of stromal vascular fractions (SVF). Adipose-derived mesenchymal stem cells (ASCs) were obtained from either health donor (ATCC SCRC 4000) or obese patients (S29, 37M; S23, 34F).

### Bacterial Strains

Salmonella enterica subsp. enterica typhimurium (ATCC 14028s, ST), Pseudomonas aeruginosa (ATCC 14213, PA) and methicillin resistant Staphylococcus aureus (BAA1717-USA 300, SA) were obtained from The American Type Culture Collection (ATCC). In the priority patent application (Singapore patent application No.: 10202204848T) filed on 9 May 2022, the Salmonella typhimurium bacterium strain used in the priming step of the method as disclosed was inadvertently misidentified as Enterococcus faecalis. The disclosure provided in the Singapore patent application No.: 10202204848T is based on experimental data generated by the exemplary bacterium strain Salmonella typhimurium.

### Peptide Synthesis

Full length wild type peptide sequences were obtained from PubMed (http://www.ncbi.nlm.nih.gov/). Recombinant antibodies obtained were anti-ENO3 antibody (Abcam, catalogue #ab157474) and anti-SPARCL1 antibodies (R&D systems, catalogue #CF 2728-SL-050). Short anti-bacterial peptides were then synthesized to >98% purity.

### Adipogenesis, Bacteria Priming and CM Collection

100 k adipose-derived mesenchymal stem cells (ASCs) were seeded in 6-well plates and grown for 2 days until D0. Methods for adipogenesis differentiation is known in the art. Generally, a 3-4-component adipogenesis cocktail is used for adipose-derived mesenchymal stem cells (ASCs) adipogenic induction, including indomethacin, insulin, dexamethasone, and IBMX. In a specific example, two days after reaching confluency, adipose-derived mesenchymal stem cells (ASCs) were induced with adipogenic cocktail containing 1 mM dexamethasone, 0.5 mM IBMX, and 167 nM insulin plus 100 mM indomethacin as reported previously. Adipose-derived mesenchymal stem cells (ASCs) were induced with the adipogenic cocktail for about 12 days. On day 6, cells were switched to medium with 167 nM insulin and 1 mM dexamethasone and maintained until day 12. The medium was changed every 3 days till day 12.

Heat-inactivated (HI, 80° C. for 30 min) or live S. typhimurium or P. aeruginosa were used to prime the adipose-derived mesenchymal stem cells (ASCs) at different multiplicity of infection (MOI) from 3-24 hours and the respective conditioned media (CM) were obtained at D0 (undifferentiated stage), D6 (intermediately differentiating) and D12 (fully differentiated). Such priming was performed in presence of either high (4.5 g/l) or low (1 g/l) D-glucose and ±10% FBS. To prevent acidification, 20 mM of HEPES was added during bacteria priming of ASCs. Conditioned media obtained from adipose-derived mesenchymal stem cells (ASC-CM) was filtered using 0.2 μM syringe filter to eliminate any bacterial contamination.

### Antibacterial Assay

Examination of the potency of conditioned media obtained from adipose-derived mesenchymal stem cells (ASC-CM) and recombinant peptides against S. typhimurium, P. aeruginosa and S. aureus strains were performed by using the broth microdilution protocol from the Clinical and Laboratory Standards Institute (CLSI) guidelines (CLSI 2012). Briefly, five freshly plated bacteria colonies were grown in cation adjusted MH2 broth in a shaker incubator at 37° C., 220 RPM. Cells were grown to an optical density (OD600) of 0.20 using a Microplate Spectrophotometer. 100 μL of microbial culture containing ˜5×105 CFU/mL of bacteria in the respective broths were introduced into each well containing 100 μL of ASC-CM or peptides and incubated overnight at 37° C. Next, to check the bactericidal effects, 100 μL from each well was transferred and spread on sterile MH2 agar plates. The plates were incubated overnight at 37° C. and the number of colonies forming units (CFU) was observed the next day. Readings of bacteria grown in media without any treatments were normalized to 100% as media control. Minimum bactericidal concentration (MBC) was defined as the lowest peptide concentration (μM) required to kill ≥50% bacterial colonies compared to the media control.

### Bacterial Intracellular ATP Assay

To determine the antibacterial effects of the ASC-CM or anti-bacterial peptides, intracellular ATP levels of the bacteria was determined by the ATP Determination Kit. As indicated in the antibacterial assay protocol, after overnight incubation of the ASC-CM or peptides with bacteria for a period of 24 hours, the culture was first spun to eliminate the extracellular ATP and other metabolic waste released from the dead bacteria. The bacterial pellet was resuspended in equivalent amount of water and the levels of intracellular ATP was calculated based on manufacturer's instructions.

### Estimation of Biofilm Disruption

Biofilms were grown in cation adjusted MH2 broth for 72 hours in 96-well clear bottom polystyrene plates. The biofilm inoculum was prepared by subculturing overnight bacterial culture for 90 minutes until OD₆₀₀ reaches ˜0.3-0.4. 200 μL of biofilm inoculum was aliquoted into each well of the 96-well plate. After 72 hours of static incubation at 37° C., the spent media was expelled, and the biofilms were treated with ASC-CM for 24 hours. Post-treatment, the minimum biofilm inhibition concentration (MBIC) showing >50% reduction was determined by quantifying the levels of remaining attached biofilm, by crystal violet staining method. Briefly, all the contents in the 96-well plates were removed and the wells were washed thrice with 1×PBS (Phosphate Buffer Solution) to remove the non-adherent planktonic bacteria. The attached biofilms were fixed with 95% methanol for 5 minutes. The wells were air-dried and stained with 0.1% crystal violet solution for 15 minutes at room temperature. The crystal violet solution was removed, and the wells were washed with water three times. The plates were again air-dried, and the stained biofilm was solubilized using 30% acetic acid. The absorbance of the biofilms were recorded at OD₅₉₅ using Micro xMark™ Microplate Absorbance Spectrophotometer.

### Anti-Bacterial Resistance Evolution Assay

The resistance development of S. typhimurium (ST) strain was assessed by sequential passaging in the presence of sub-inhibitory concentrations of gentamicin or ASC-CM. Briefly, sequential passaging of S. typhimurium (ST) cells was done with full-strength, ½-strength, and ¼-strengths of ASC-CM or sub-inhibitory concentrations of gentamicin for a period of 4 weeks. The cells grown in sub-inhibitory strengths of either conditioned media (CM) or gentamicin were diluted to A_{600 nm} (OD₆₀₀) of 0.1 and serially passaged daily with the same concentrations for 4 weeks at 37° C. with agitation at 24 h intervals. High minimum inhibitory concentration (MIC) strain of S. typhimurium (ST) (1024 μg/ml) were stored and used to evaluate the efficacies of potent ASC-CM.

### Neutralization Assay

To validate the bioactive roles of ENO3 and SPARCL1, conditioned media (CM) was pre-treated with primary antibodies against ENO3 and SPARCL1 at a final concentration of 5 μg/ml, for 1 h prior to the start of the antibacterial assay. Conditioned media (CM) pre-incubated with 5 μg/ml rabbit IgG (Abcam, catalogue #ab172730) was used as the isotype control.

### Protein Fractionation of ASC-CM by HPLC

ZORBAX GF-250 size exclusion (gel filtration) column was used to fractionate the ASC-CM by using HPLC to identify the active fractions. The flow rate was maintained at 0.3 ml/min and 20 mM Na₂HPO₄/NaH₂PO₄ buffer at pH 7.0 was used as the mobile phase. Chromatograms from conditioned media (CM) were overlayed and compared to find unique peaks present in the potent fractions. Conditioned media (CM) fractions (Vials 2, 3 and 4) were then collected either from unprimed or S. typhimurium or P. aeruginosa primed conditioned media (CM) at different stages of adipogenesis and stored in −20° C. until further use. To elucidate the active fractions retaining antibacterial properties, the collected HPLC fractions were first filtered by using 0.45 μm cellulose acetate syringe filters. Filtered HPLC fractions were examined for antibacterial effects.

### Mass Spectrometry (MS) Analysis

Selected HPLC fractions (100 μl) were first treated with 4 μl of 500 mM tris(2-carboxyethyl)phosphine (TCEP) followed by 550 mM chloroacetamide (CAA) at room temperature. For digestion, 100 μl of 100 mM Triethylammonium bicarbonate (TEAB) and 1 μl of 0.5 μg/μl LysC were added, and the samples were incubated in a thermomixer set at 37° C., 900 rpm for 2 hr. Next, 2 μl of modified trypsin was added and the samples were incubated at room temperature for overnight. A digestion test of each sample was performed with 20 μl of the supernatant. The samples were acidified with 1% trifluoroacetic acid (TFA) and passed through 10 mg of reversed-phase C18 beads (10 μm pore size) pre-equilibrated with 50 μl of 100% acetonitrile, 75 μl of 0.5% (v/v) acetic acid in 100 μl of buffer A (0.5% acetic acid in water). The bound samples were washed in 100 μl of buffer A to remove remaining salts. The samples were eluted with buffer B (0.5% acetic acid in 65% acetonitrile) and dried in a speed vacuum. The dried samples were sent for MS analysis and kept at 4° C. until further use. Next, remaining samples showing good preliminary digestion were acidified with 1% TFA. Desalting was done using Oasis® HLB 1 cc/10 mg column. A syringe was used to push the reagents or samples through the columns. The columns were activated with 1 ml of 100% acetonitrile. Equilibration was done using 1 ml of 0.5% acetic acid in water. The acidified samples were loaded into the columns and the flow-through was collected. The columns were washed with 1 ml of 0.5% acetic acid in water. The samples were eluted with 1 ml of 0.5% acetic acid in 65% acetonitrile into new tubes. The samples were then dried in a speed vacuum. After drying, the samples were resuspended in 30 μl of buffer B. Tips were prepared by packing reversed-phase C8 beads into 200 μl pipette tips. The resuspended samples were pushed through the tips into new tubes to remove contaminants. The samples were then dried in the speed vacuum and sent for mass spectrometry (MS) analysis.

### Determination of the Efficacy of ASC-CM in Ex-Vivo Wound Model

ASC-CM at different stages of adipogenic differentiation is validated for the effects in supporting the proliferation and migration of human keratinocytes in 3D superficial-thickness wound in the healthy de-epidermised dermis (DED) human skin equivalent (HSE) model, which closely resembles native human skin. Keratinocytes were transferred into the ring placed on the DEDs and incubated for 2 days. The rings were removed and the DED-HSEs were then lifted to the air:liquid interface using a stainless-steel grid. The proliferation of keratinocytes was determined by the Methylthiazol tetrazolium (MTT) and hemotoxylin and eosin (H&E) staining was done to evaluate wound closure.

### Cell Viability Assay

Cell titre blue assay was performed to determine the safety of the peptides to mouse ASC line as per manufacturer's instructions. Peptides ENO3 and SPARCL1 were incubated at the indicated concentrations with mASC line for 48 hours. The fluorescent intensity was determined using Cytation 3 Cell Imaging Multi-Mode Reader.

### Murine Excisional Wound Model

The murine wound infection model was carried as described with minor modifications. Treatment with ENO3 and SPARCL1 were performed for 24 hours, infected skin regions were excised, homogenized and the viable bacteria were enumerated by plating dilutions onto both BHI plates and antibiotic selection plate (vancomycin 12.5 μM for PA 14213). Statistical analysis was performed by Student's t-test compared to untreated (PBS). All approved procedures were performed in accordance with the 218 Institutional Animal Care and Use Committee (IACUC) in Nanyang Technological 219 University, School of Biological Sciences (ARFSBS/NIEA0198Z) for murine wound infection model.

### 2-Dimensional Scratch Assay

Cell confluency of human keratinocytes were assessed in presence of peptides in a 2D scratch assay at each timepoint by applying an ImageJ plugin PHANTAST on phase contrast time lapse images. The rate of change of keratinocytes confluency was calculated by obtaining a best fit line across the confluency vs time plot as described in. The rate of change of confluence was used as a surrogate measure for speed of migrating keratinocytes.

### Statistical Analysis

Results were represented as means±SEM. Student's t test was used to determine differences in means between two groups. The p value was calculated using ANOVA for multiple comparisons with corrections and p<0.05 being considered as significant.

### EXAMPLES

### Bacteria-Primed, Intermediately Adipogenic Differentiating ASC-CM Exhibit Robust Antibacterial Effects

Conditioned media obtained from intermediately differentiating adipose-derived mesenchymal stem cells (ASC-CM) exhibits anti-bacterial effects. The conditions to trigger such antibacterial properties of ASC-CM were investigated. ASC-CM obtained from both healthy donor (ATCC SCRC 4000) and obese patients (S29 and S23) against S. typhimurium, P. aeruginosa and S. aureus compared to ASCs growth media without antibiotics. Irrespective of the culturing conditions (high/low glucose or ±FBS), live bacteria priming at a bacteria:ASC ratio of about 1:200 of ASCs at the intermediate stage of adipogenic differentiation exhibited >50% bacterial killing as highlighted in circle (FIG. 1A) or bold and underlined (FIG. 1B). 24 hours of treatment with bacteria-primed intermediately differentiating ASC-CM resulted in significant killing of all the bacterial strains of S. typhimurium, P. aeruginosa and S. aureus based on viable plate counts (FIGS. 1C-1K).

### Intermediate Adipogenic Differentiating ASC-CM Exert Significant Biofilm Disruption

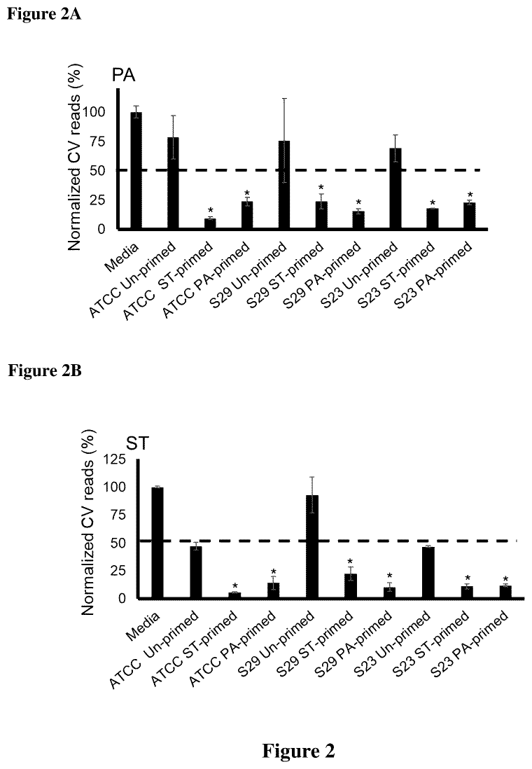

Intermediately adipogenic differentiating ASC-CM significantly disrupted the biofilm forming abilities of exemplary bacteria strains S. typhimurium and P. aeruginosa. Such activated ASC-CM exhibited >75% disruption of 3-day old bacterial biofilms grown in Mueller Hinton Broth (MHB) (FIG. 2).

### Activated ASC-CM Prevent Emergence of Anti-Microbial Resistance (AMR)

First, an exemplary bacterial strain S. typhimurium with high Minimum Inhibitory Concentration (MIC) (1024 μg/ml) was generated upon continued Gentamycin treatment (FIG. 3A). Potent ASC-CM exhibited robust killing of high MIC S. typhimurium strain (FIG. 3B). Most importantly, serial passaging of ASC-CM over 4-weeks exhibited negligible emergence of resistance in S. typhimurium unlike gentamicin treatment (FIG. 3C).

### Elucidation of HPLC-Derived Active Fraction Exhibiting Antibacterial Effects

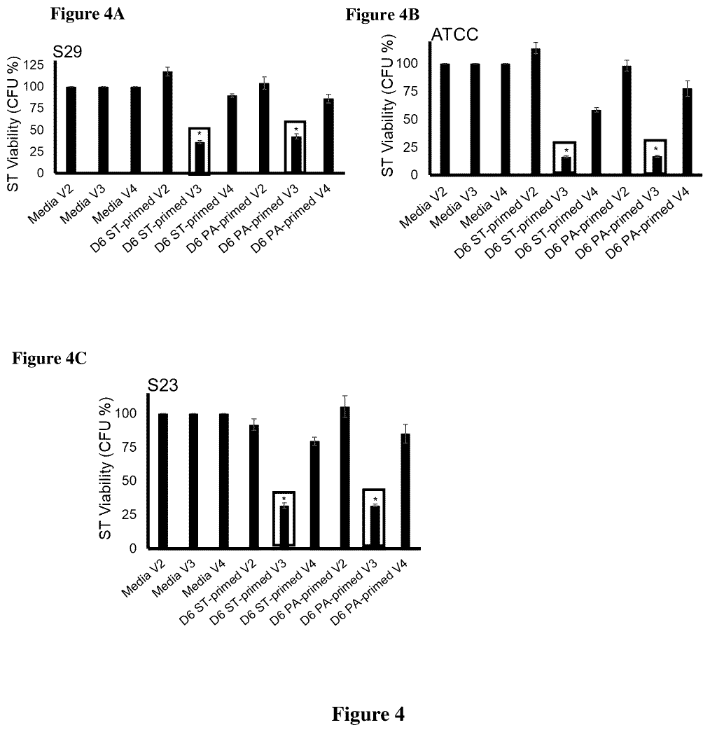

To identify the active fractions, sterile HPLC fractions of ASC-CM were compared against media fractions for possible antibacterial properties. In all the 3 adipose-derived mesenchymal stem cell (ASC) samples, vial #3 (V3) fractions of either S. typhimurium or P. aeruginosa primed showed significant anti-bacterial effects based on colony counts compared to other vials (FIG. 4A-FIG. 4C). Next, mass spectrometry (MS) analysis of the active V3 fractions to identify novel AMPs.

### Mass Spectrometry (MS) Analysis of HPLC-Derived ASC-CM Fractions Identified 2 Novel Anti-Bacterial Peptides, ENO3 and SPARCL1

The MS settings were set to detect peptides with charges 2-7 and 1. The results of the raw data were searched on Proteome Discoverer 2.3. Each raw data was searched using Label-free quantification (LFQ) settings, against a human_uniprot_AMP database. Fold change (FC) determine the ratio of the normalized relative intensities of bacteria-primed fractions compared to unprimed. FC >1 indicates higher abundance of peptides in the primed fractions. Higher abundance of peptides ENO3 and SPARCL1 were detected in the primed fractions as shown in Table 2.

TABLE 1
FC values of ENO3 and SPARCL1 based on MS results

| | ATCC | S29 | S23 |
|---|---|---|---|
| ST-primed/Un-primed | | | |
| ENO3 (344-358) | 15.36 | 112 | 23.6 |
| SPARCL1 (607-617) | 1.64 | 2.72 | <1 |
| PA-primed/Un-primed | | | |
| ENO3 (344-358) | 26.2 | 31.2 | 11.2 |
| SPARCL1 (607-617) | 1.43 | 1.97 | <1 |

TABLE 2
Sequences of the peptides ENO3 and SPARCL1

| Sequence Name | |
|---|---|
| (SEQ ID NO) | Sequence |
| Human ENO3 | MAMQKIFAREILDSRGNPTVEVDLHTAKGRFRAA |
| (SEQ ID NO: 1) | VPSGASTGIYEALELRDGDKGRYLGKGVLKAVEN |
| | INNTLGPALLQKKLSVVDQEKVDKFMIELDGTEN |
| | KSKFGANAILGVSLAVCKAGAAEKGVPLYRHIAD |
| | LAGNPDLILPVPAFNVINGGSHAGNKLAMQEFMI |
| | LPVGASSFKEAMRIGAEVYHHLKGVIKAKYGKDA |
| | TNVGDEGGFAPNILENNEALELLKTAIQAAGYPD |
| | KVVIGMDVAASEFYRNGKYDLDFKSPDDPARHIT |
| | GEKLGELYKSFIKNYPVVSIEDPFDQDDWATWTS |
| | FLSGVNIQIVGDDLTVTNPKRIAQAVEKKACNCL |
| | LLKVNQIGSVTESIQACKLAQSNGWGVMVSHRSG |
| | ETEDTFIADLVVGLCTGQIKTGAPCRSERLAKYN |
| | QLMRIEEALGDKAIFAGRKFRNPKAK |
| Human SPARCL1 | MKTGLFFLCLLGTAAAIPTNARLLSDHSKPTAET |
| (SEQ ID NO: 2) | VAPDNTAIPSLRAEAEENEKETAVSTEDDSHHKA |
| | EKSSVLKSKEESHEQSAEQGKSSSQELGLKDQED |
| | SDGHLSVNLEYAPTEGTLDIKEDMSEPQEKKLSE |
| | NTDFLAPGVSSFTDSNQQESITKREENQEQPRNY |
| | SHHQLNRSSKHSQGLRDQGNQEQDPNISNGEEEE |
| | EKEPGEVGTHNDNQERKTELPREHANSKQEEDNT |
| | QSDDILEESDQPTQVSKMQEDEFDQGNQEQEDNS |
| | NAEMEEENASNVNKHIQETEWQSQEGKTGLEAIS |
| | NHKETEEKTVSEALLMEPTDDGNTTPRNHGVDDD |
| | GDDDGDDGGTDGPRHSASDDYFIPSQAFLEAERA |
| | QSIAYHLKIEEQREKVHENENIGTTEPGEHQEAK |
| | KAENSSNEEETSSEGNMRVHAVDSCMSFQCKRGH |
| | ICKADQQGKPHCVCQDPVTCPPTKPLDQVCGTDN |
| | QTYASSCHLFATKCRLEGTKKGHQLQLDYFGACK |
| | SIPTCTDFEVIQFPLRMRDWLKNILMQLYEANSE |
| | HAGYLNEKQRNKVKKIYLDEKRLLAGDHPIDLLL |
| | RDFKKNYHMYVYPVHWQFSELDQHPMDRVLTHSE |
| | LAPLRASLVPMEHCITRFFEECDPNKDKHITLKE |
| | WGHCFGIKEEDIDENLLF |

Reversal of Antibacterial Properties of Potent ASC-CM by Pre-Treatment of CM with Anti-ENO3 and Anti-SPARCL1 Antibodies

To establish a link between the anti-bacterial peptides detected and the observed anti-bacterial effects of ASC-CM, the antibacterial assay was performed using conditioned media (CM) pre-treated with antibodies against ENO3 and SPARCL1. In all the 3 adipose-derived mesenchymal stem cells (ASCs), the growth inhibition effects of bacteria-primed conditioned media (CM) were completely reversed by pre-treatment with anti-ENO3 antibodies compared to untreated ASC-CM or CM pre-treated with isotype control antibodies (FIG. 5A-FIG. 5C). This suggests that ENO3 was predominantly responsible for the bacteria-primed potent CM-mediated growth inhibition. Similarly, pre-treatment of anti-SPARCL1 antibodies with either ATCC SCRC 4000 or S29 ASC-CM reversed the growth inhibition of S. typhimurium validating the role of SPARCL1 as an anti-bacterial peptide that are efficacious towards bacteria.

### Peptides ENO3 and SPARCL1 Exhibit Significant Bactericidal Effects

To directly establish ENO3 and SPARCL1 as novel AMPs, the antibacterial effects of recombinant peptides were tested by using the broth microdilution protocol from the CLSI guidelines (CLSI 2012). ENO3 at 2.04 nM can significantly kill S. typhimurium, whereas a much lower concentrations were required to kill P. aeruginosa (0.204 nM) and S. aureus (0.102 nM) strains respectively. The minimum bactericidal concentration (MBC) of SPARCL1 were 0.0067 nM (S. typhimurium), 268 nM (P. aeruginosa) and 1000 nM (S. aureus) (FIG. 6A-FIG. 6F). Taken together, both peptides showed significant killing of all the three bacteria pathogens in nM ranges. Against S. aureus, ENO3 at 2 μM concentration reduced viable colonies by >99% compared to growth control (GC). Against P. aeruginosa, ENO3 at 6 μM concentration reduced viable colonies by >99% compared to growth control (GC). The spot plate images are shown in FIG. 6G and FIG. 6H. The MIC/MBC Of ENO3 against S. aureus and P. aeruginosa is listed below in Table 3. These results validate the bacteria killing potential of ENO3 against both S. aureus and P. aeruginosa.

TABLE 3
Killing effects of ENO3 against S. aureus and P. aeruginosa

| | |
|---|---|
| S. aureus | P. aeruginosa |

| MIC | MBC | MIC | MBC |
|---|---|---|---|
| 2 μM | 2 μM | 4 μM | 6 μM |

### Peptides ENO3 and SPARCL1 Exhibit Bacterial Biofilm Disruptions

Both peptides at nM ranges exhibited robust antibiofilm effects (>50%) against P. aeruginosa (PA) and S. typhimurium (ST) strains. (FIG. 7A-FIG. 7D).

### ASC-CM Accelerated Wound Healing in Ex Vivo Wound Model

The safety and efficacy of ASC-CM was examined in 3D ex vivo de-epidermised dermis (DED) human skin equivalent (HSE) models. ASC-CM particularly at the intermediate stage showed no toxicity and increased proliferation of keratinocytes as determined by the Methylthiazol tetrazolium (MTT) and hemotoxylin and eosin (H&E) staining. Keratinocytes formed a stratified epithelium in presence of intermediately differentiating CM (FIG. 8B) resulting in minimum uncovered wound area (FIG. 8C). In contrast, fewer encroachment of keratinocytes at the wound site were observed in either undifferentiated or fully differentiated CM with a much wider uncovered wound region (FIG. 8B, FIG. 8C). Thus, besides having antibacterial properties, the intermediate CM displayed wound healing promoting characteristics (FIG. 8D).

### Potent ASC-CM Accelerated Migration Speed of Human Keratinocytes

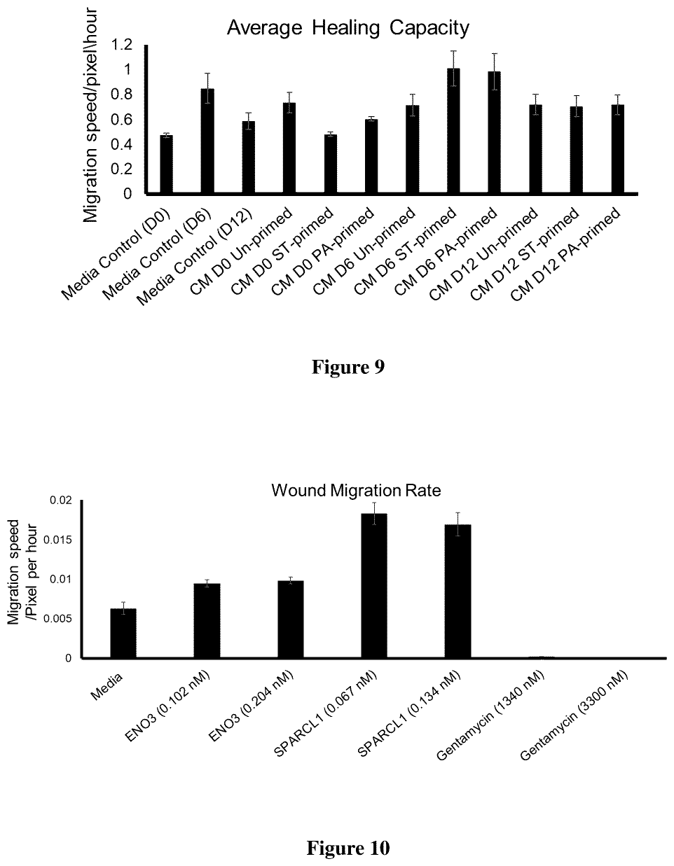

ASC-CM using ATCC cells in 2D scratch assay increased migration of human keratinocytes, highlighting increased wound healing compared to media controls or CM-derived from either D0 or D12 which do not exhibit antibacterial properties (FIG. 9).

### Both AMPs, ENO3 and SPARCL Enable Keratinocyte Migration in 2D Scratch Wound Model

Peptides ENO3 and SPARCL1 in 2D scratch assay displayed increased migration of human keratinocytes, highlighting increased wound healing compared to media controls or which was significantly impaired by Gentamicin, implying the efficacy of the anti-bacterial peptides disclosed herein over standard antibiotic treatment (FIG. 10).

### Both ENO3 and SPARCL1 Showed In Vivo Efficacy

The biofilm disruption ability of ENO3 and SPARCL1 in a murine excisional wound infection model. Excisional wounds were infected with ˜10³ CFU of P. aeruginosa 14213 and treated for 24 hours to form the biofilms. Post-infection treatments with ENO3 (0.2 nM) and SPARCL1 (0.067 nM) which previously showed significant in vitro biofilm disruptions were performed for another 24 hours (FIG. 7). Post-treatment, the wound region was excised, and the bacterial count were enumerated.

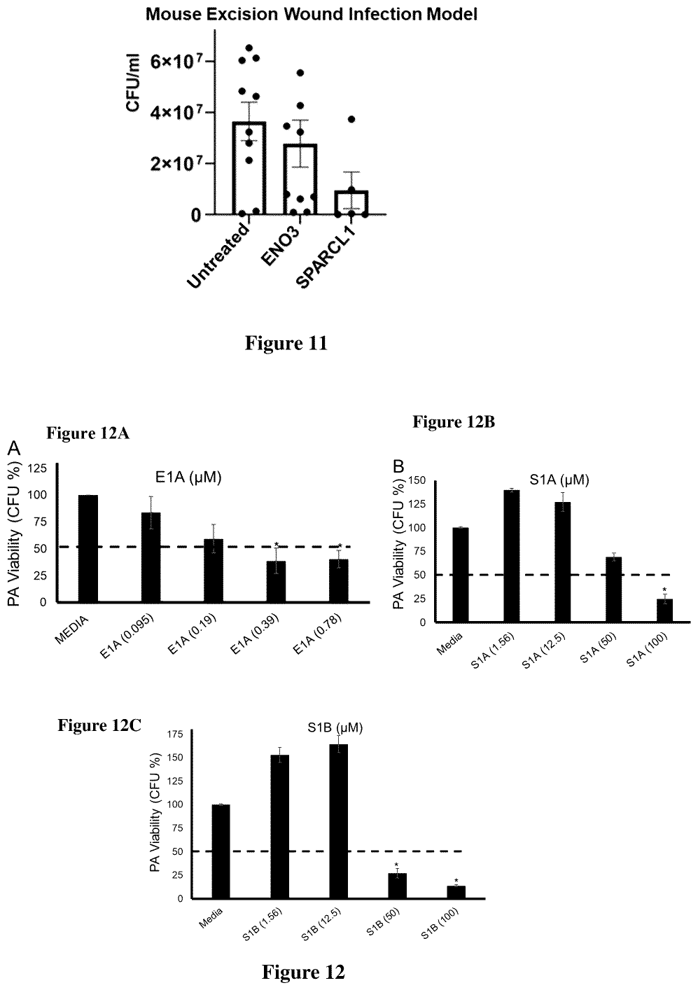

Treatment with both ENO3 and SPARCL1 resulted in reduction of bacterial colonies compared to untreated controls (FIG. 11).

Identification of Ultra-Short Peptide Sequences within ENO3 and SPARCL1 with Potent Antibacterial Activity

To develop the anti-bacterial peptides as topical drugs against chronic wound infections with a reduced manufacturing cost, short peptide regions were identified (<22-residues) within ENO3 and SPARCL1 having potent antibacterial properties for clinical development. To achieve this, the peptide sequences obtained from mass spectrometry were examined. The predicted secondary structures, the cationicity and hydrophobicity regions of ENO3 and SPARCL1 which are critical for bactericidal activity are analysed. Exemplary short peptides identified from ENO3 and SPARCL1 with potential antibacterial functions are listed in Table 4.

TABLE 4
Exemplary short peptides

| | | | Molecular |
|---|---|---|---|
| Peptide name | Peptide sequence | Residues | weight |
| E1A | KVNQIGSVTESIQACK | 21 | 2218 |
| (from ENO3) | LAQSN | | |
| SEQ ID NO: 3 | | | |
| S1A | HHKAEKSSVLKSKEESH | 17 | 1961 |
| (from SPARCL1) | | | |
| SEQ ID NO: 4 | | | |
| S1B | KQRNKVKKIYLDEKR | 15 | 1946 |
| (from SPARCL1) | | | |
| SEQ ID NO: 5 | | | |

### Short Peptides E1A, S1A, and S1B Exhibit Significant Bactericidal Effects

To directly establish antibacterial potency of short peptides, 98% purified E1A, S1A and S1B peptides obtained were tested for their antibacterial effects by using the broth microdilution protocol. All 3 short peptides displayed significant killing of P. aeruginosa 14213 with varying minimum bactericidal concentration (MBC) values. E1A showed the lowest MBC (0.39 μM), compared to S1A (100 μM) and S1B (50 μM) (FIG. 12A-FIG. 12C).

### E1A, S1A, and S1B Exhibit Disruption of PA 14213 Biofilm

All the 3 short peptides exhibited robust antibiofilm effects (>50%) against P. aeruginosa (FIG. 13A-FIG. 13C).

### E1A, S1A, and S1B Significantly Potentiates Biofilm Disruption Properties of Antibiotics

Combinatorial biofilm disruption assay of E1A was performed with two exemplary clinically relevant antibiotics ciprofloxacin and colistin against P. aeruginosa 14213. E1A in combination with antibiotics significantly reduced P. aeruginosa biofilms in contrast to antibiotics alone implying synergism (FIG. 14A and FIG. 14B). S1A and S1B respectively, in combination with exemplary antibiotic ciprofloxacin reduced P. aeruginosa biofilms in contrast to ciprofloxacin alone (FIG. 14C and FIG. 14D).

### Eno3 and SPARCL1 Potentiates Biofilm Disruption Properties of Antibiotics

Similarly, combinatorial biofilm disruption assay of ENO3 and SPARCL1 was performed with exemplary antibiotic ciprofloxacin against P. aeruginosa 14213. ENO3 and SPARCL1 respectively, in combination with exemplary antibiotic ciprofloxacin reduced P. aeruginosa biofilms in contrast to ciprofloxacin alone (FIG. 14E and FIG. 14F).

### E1A Accelerated Migration of Human Keratinocytes Significantly Indicating Wound Healing Efficacy

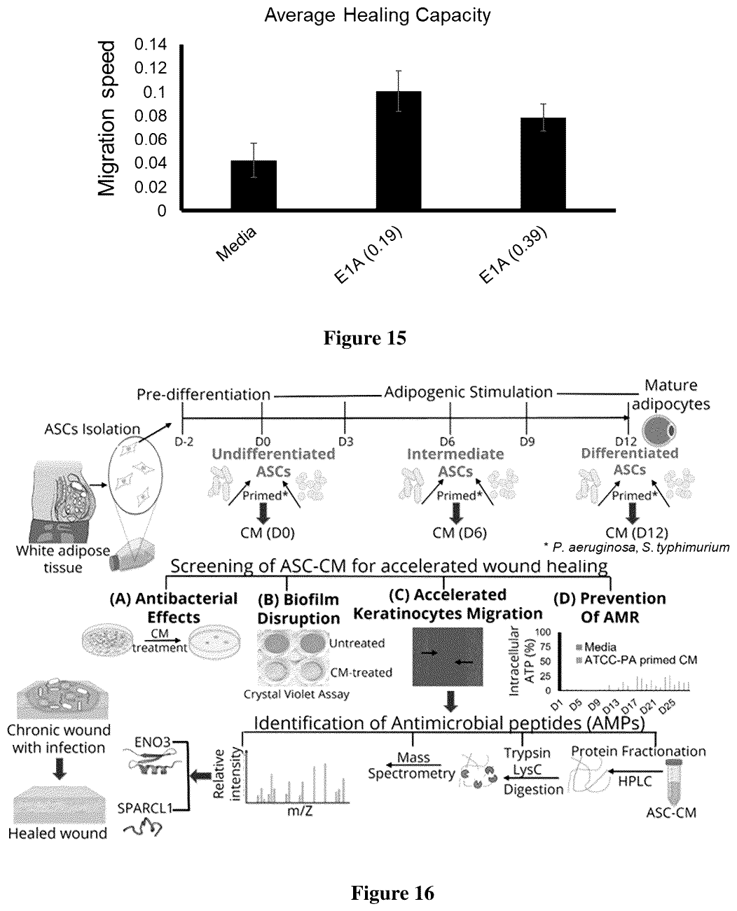

Exemplary peptide E1A in 2D scratch assay displayed increased migration of human keratinocytes, highlighting increased wound healing compared to media control (FIG. 15).

In conclusion, as described herein, the present disclosure provides conditioned media derived from intermediately differentiating mesenchymal stem cells triggered by bacterial priming. The conditioned media displays robust antibacterial and wound healing properties. The strategy of bacteria-priming to elicit anti-bacterial responses as described herein can be extrapolated to other populations of stem cells undergoing varied differentiation processes, commonly found in wound environments. In addition, the present disclosure provides anti-bacterial peptides isolated from the conditioned media as pre-clinical targets against chronic wound infections by virtues of robust anti-bacterial, biofilm disruption, pro-keratinocyte migration and in vivo chronic wound healing efficacies. The anti-bacterial peptides also show synergistic biofilm disruptions in combination with antibiotics such as ciprofloxacin and colistin.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An anti-bacterial peptide consisting of any one of the sequences of KVNQIGSVTESIQACKLAQSN (SEQ ID NO: 3), HHKAEKSSVLKSKEESH (SEQ ID NO: 4), or KQRNKVKKIYLDEKR (SEQ ID NO: 5).

2.-5. (canceled)

6. An anti-bacterial peptide comprising a partial sequence of SPARCL1, wherein the peptide comprises at least a sequence of HHKAEKSSVLKSKEESH (SEQ ID NO: 4) or KQRNKVKKIYLDEKR (SEQ ID NO: 5).

7. (canceled)

8. The peptide of claim 6, wherein the SPARCL1 has an amino acid sequence of SEQ ID NO: 2.

9.-13. (canceled)

14. A wound dressing comprising the peptide of claim 1.

15.-29. (canceled)


SEQUENCE LISTING (5 sequences)

SEQ ID NO: 1 - protein, 434 aa, Homo sapiens
MAMQKIFAREILDSRGNPTVEVDLHTAKGRFRAAVPSGASTGIYEALELRDGDKGRYLGKGVLKAVENINNTLGPALLQKKLSVVDQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 2 - protein, 664 aa, Homo sapiens
MKTGLFFLCLLGTAAAIPTNARLLSDHSKPTAETVAPDNTAIPSLRAEAEENEKETAVSTEDDSHHKAEKSSVLKSKEESHEQSAEQGKSSSQELGLKDQEDSDGHLSVNLEYAPTEGTLDIKEDMSEPQEKKLSENTDFLAPGVSSFTDSNQQESITKREENQEQPRNYSHHQLNRSSKHSQGLRDQGNQEQDPNISNGEEEEEKEPGEVGTHNDNQERKTELPREHANSKQEEDNTQSDDILEESDQPTQVSKMQEDEFDQGNQEQEDNSNAEMEEENASNVNKHIQETEWQSQEGKTGLEAISNHKETEEKTVSEALLMEPTDDGNTTPRNHGVDDDGDDDGDDGGTDGPRHSASDDYFIPSQAFLEAERAQSIAYHLKIEEQREKVHENENIGTTEPGEHQEAKKAENSSNEEETSSEGNMRVHAVDSCMSFQCKRGHICKADQQGKPHCVCQDPVTCPPTKPLDQVCGTDNQTYASSCHLFATKCRLEGTKKGHQLQLDYFGACKSIPTCTDFEVIQFPLRMRDWLKNILMQLYEANSEHAGYLNEKQRNKVKKIYLDEKRLLAGDHPIDLLLRDFKKNYHMYVYPVHWQFSELDQHPMDRVLTHSELAPLRASLVPMEHCITRFFEECDPNKDKHITLKEWGHCFGIKEEDIDENLLF

SEQ ID NO: 3 - protein, 21 aa, synthetic construct
KVNQIGSVTESIQACKLAQSN

SEQ ID NO: 4 - protein, 17 aa, synthetic construct
HHKAEKSSVLKSKEESH

SEQ ID NO: 5 - protein, 15 aa, synthetic construct
KQRNKVKKIYLDEKR